SEQUENCE LISTING (58 sequences)

SEQ ID NO: 1 - DNA, 4281 bp, Artificial Sequence
  synthetically generated
atggcttctagcgactacaaggaccacgacggggactacaaggaccacgacatcgactacaaggacgacgacgacaagatggctccaaagaagaagaggaaggttggcatccacggggtgccggctgctgacaagaagtactcgatcggcctcgacatcgggacgaactcagttggctgggccgtgatcaccgacgagtacaaggtgccctctaagaagttcaaggtcctggggaacaccgaccgccattccatcaagaagaacctcatcggcgctctcctgttcgacagcggggagaccgctgaggctacgaggctcaagagaaccgctaggcgccggtacacgagaaggaagaacaggatctgctacctccaagagattttctccaacgagatggccaaggttgacgattcattcttccaccgcctggaggagtctttcctcgtggaggaggataagaagcacgagcggcatcccatcttcggcaacatcgtggacgaggttgcctaccacgagaagtaccctacgatctaccatctgcggaagaagctcgtggactccaccgataaggcggacctcagactgatctacctcgctctggcccacatgatcaagttccgcggccatttcctgatcgagggggatctcaacccagacaacagcgatgttgacaagctgttcatccaactcgtgcagacctacaaccaactcttcgaggagaacccgatcaacgcctctggcgtggacgcgaaggctatcctgtccgcgaggctctcgaagtccaggaggctggagaacctgatcgctcagctcccaggcgagaagaagaacggcctgttcgggaacctcatcgctctcagcctggggctcaccccgaacttcaagtcgaacttcgatctcgctgaggacgccaagctgcaactctccaaggacacctacgacgatgacctcgataacctcctggcccagatcggcgatcaatacgcggacctgttcctcgctgccaagaacctgtcggacgccatcctcctgtcagatatcctccgcgtgaacaccgagatcacgaaggctccactctctgcctccatgatcaagcgctacgacgagcaccatcaggatctgaccctcctgaaggcgctggtccgccaacagctcccggagaagtacaaggagattttcttcgatcagtcgaagaacggctacgctgggtacatcgacggcggggcctcacaagaggagttctacaagttcatcaagccaatcctggagaagatggacggcacggaggagctcctggtgaagctcaacagggaggacctcctgcggaagcagagaaccttcgataacggcagcatcccccaccaaatccatctcggggagctgcacgccatcctgagaaggcaagaggacttctaccctttcctcaaggataaccgggagaagatcgagaagatcctgaccttcagaatcccatactacgtcggccctctcgcgcgggggaactcaagattcgcttggatgacccgcaagtctgaggagaccatcacgccgtggaacttcgaggaggtggtggacaagggcgctagcgctcagtcgttcatcgagaggatgaccaacttcgacaagaacctgcccaacgagaaggtgctccctaagcactcgctcctgtacgagtacttcaccgtctacaacgagctcacgaaggtgaagtacgtcaccgagggcatgcgcaagccagcgttcctgtccggggagcagaagaaggctatcgtggacctcctgttcaagaccaaccggaaggtcacggttaagcaactcaaggaggactacttcaagaagatcgagtgcttcgattcggtcgagatcagcggcgttgaggaccgcttcaacgccagcctcgggacctaccacgatctcctgaagatcatcaaggataaggacttcctggacaacgaggagaacgaggatatcctggaggacatcgtgctgaccctcacgctgttcgaggacagggagatgatcgaggagcgcctgaagacgtacgcccatctcttcgatgacaaggtcatgaagcaactcaagcgccggagatacaccggctgggggaggctgtcccgcaagctcatcaacggcatccgggacaagcagtccgggaagaccatcctcgacttcctgaagagcgatggcttcgccaacaggaacttcatgcaactgatccacgatgacagcctcaccttcaaggaggatatccaaaaggctcaagtgagcggccagggggactcgctgcacgagcatatcgcgaacctcgctggctcccccgcgatcaagaagggcatcctccagaccgtgaaggttgtggacgagctcgtgaaggtcatgggccggcacaagcctgagaacatcgtcatcgagatggccagagagaaccaaaccacgcagaaggggcaaaagaactctagggagcgcatgaagcgcatcgaggagggcatcaaggagctggggtcccaaatcctcaaggagcacccagtggagaacacccaactgcagaacgagaagctctacctgtactacctccagaacggcagggatatgtacgtggaccaagagctggatatcaaccgcctcagcgattacgacgtcgatcatatcgttccccagtctttcctgaaggatgactccatcgacaacaaggtcctcaccaggtcggacaagaaccgcggcaagtcagataacgttccatctgaggaggtcgttaagaagatgaagaactactggaggcagctcctgaacgccaagctgatcacgcaaaggaagttcgacaacctcaccaaggctgagagaggcgggctctcagagctggacaaggccggcttcatcaagcggcagctggtcgagaccagacaaatcacgaagcacgttgcgcaaatcctcgactctcggatgaacacgaagtacgatgagaacgacaagctgatcagggaggttaaggtgatcaccctgaagtctaagctcgtctccgacttcaggaaggatttccagttctacaaggttcgcgagatcaacaactaccaccatgcccatgacgcttacctcaacgctgtggtcggcaccgctctgatcaagaagtacccaaagctggagtccgagttcgtgtacggggactacaaggtttacgatgtgcgcaagatgatcgccaagtcggagcaagagatcggcaaggctaccgccaagtacttcttctactcaaacatcatgaacttcttcaagaccgagatcacgctggccaacggcgagatccggaagagaccgctcatcgagaccaacggcgagacgggggagatcgtgtgggacaagggcagggatttcgcgaccgtccgcaaggttctctccatgccccaggtgaacatcgtcaagaagaccgaggtccaaacgggcgggttctcaaaggagtctatcctgcctaagcggaacagcgacaagctcatcgccagaaagaaggactgggacccaaagaagtacggcgggttcgacagccctaccgtggcctactcggtcctggttgtggcgaaggttgagaagggcaagtccaagaagctcaagagcgtgaaggagctcctggggatcaccatcatggagaggtccagcttcgagaagaacccaatcgacttcctggaggccaagggctacaaggaggtgaagaaggacctgatcatcaagctcccgaagtactctctcttcgagctggagaacggcaggaagagaatgctggcttccgctggcgagctccagaaggggaacgagctcgcgctgccaagcaagtacgtgaacttcctctacctggcttcccactacgagaagctcaagggcagcccggaggacaacgagcaaaagcagctgttcgtcgagcagcacaagcattacctcgacgagatcatcgagcaaatctccgagttcagcaagcgcgtgatcctcgccgacgcgaacctggataaggtcctctccgcctacaacaagcaccgggacaagcccatcagagagcaagcggagaacatcatccatctcttcaccctgacgaacctcggcgctcctgctgctttcaagtacttcgacaccacgatcgatcggaagagatacacctccacgaaggaggtcctggacgcgaccctcatccaccagtcgatcaccggcctgtacgagacgaggatcgacctctcacaactcggcggggataagagacccgcagcaaccaagaaggcagggcaagcaaagaagaagaagtga

SEQ ID NO: 2 - DNA, 467 bp, Artificial Sequence
  synthetically generated
gaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttggagacggagtcgattcgtctcgttttagagctagaaatagcaagttaaaataaggctagtccgttatcaacttgaaaaagtggcaccgagtcggtgcttttttt

SEQ ID NO: 3 - DNA, 25 bp, Artificial Sequence
  synthetically generated
  misc_feature (6)..(24): n is a, c, g, or t
acttgnnnnnnnnnnnnnnnnnnng

SEQ ID NO: 4 - DNA, 25 bp, Artificial Sequence
  synthetically generated
  misc_feature (6)..(24): n is a, c, g, or t
aaaacnnnnnnnnnnnnnnnnnnnc

SEQ ID NO: 5 - DNA, 2579 bp, Artificial Sequence
  synthetically generated
tggtgctactggagctgctagtggcaggccagcaggtttatttggggctggacttccggaattagatcaaatgcagcaacagttgagccagaatcccaaccttatgagggagataatgaacatgccaatgatgcagagtctcatgaataaccctgatctaatacgcaatatgattatgaataatccacaaatgcgtgatattattgatcggaatccagatcttgcccatgtcctcaatgatcctagtgttctccgccagacccttgaagctgcaagaaaccctgaaattatgagggagatgatgcggaacacagacagagcaatgagcaacatcgaagcttcccctgaagggtttaatatgctccggcgtatgtatgaaactgtacaggagccttttcttaatgcaacaacaatgggagggggtggggaaggcaccccggcctctaacccgtttgcagctcttcttggaaatcaggggcctaaccaagccggcaatgctccaactaccggcccagagtccacaacaggaacccctgttccaaatactaatccacttccaaacccctggagcaacaatggtaggttctagttatttagagttttttgtttgttttgttgttgaatgttgataattacatgtggtagtatttttattctcacagctgctgataattgcctgtgatactattatattttcccagctgggggtgcgcaaggaacaacacggtcaggtcctgctgctagtccagagggcagaggaagtcttctaacatgcggtgacgtggaggagaatcccgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaaagcggccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgcagggctggtgcaactggtggcccaccagggctgggttcagcagatttgagcagcctgctcggtggtcttggtgggaatgcaagaactggtgctgcaggtggtctaggagggttgggttcagcagatttggggagtatgcttggtggtccacctgatgctgctcttttgagtcagatgctgcaaaaccctgctatgatgcagatgatgcagaacattatgtctgacccacagtcaatgaaccaggtccaatatttttcaaaactagttcttttatgatttttggagatgaccttggatcattctgtaacatttgcttgtcccacagttgcttagcatgaacccaaatgcacgtagcctgatggagtcaaacactcagttgagggatatgttccaaaacccagaatttcttcgccagatggcatccccagaggctttgcaggtaaaatctgttgtgatgcaagttaacaactgttctcgtattttattttctgataaaatttgtatttgttctgcgcagcaattactctcattccagcagacactgtcatcacagcttggccaaaatcaacctagccagtgagtaactcttttttttgcgagaaaaaagggaaaaagtaacactctaattcaatagcatgattgtatcaccccttttttttatgaaattaaataaaatagagattatgaagtgcagttatgtttatcttttgagggtgcaattatgcgtttgctgagtcttttcttttcagggctggtaacctagggggcaatg

SEQ ID NO: 6 - DNA, 2348 bp, Artificial Sequence
  synthetically generated
gcctcaaccagaatcagcagctcagctcatggcttcctctgctcccttggtgcacctgcagtggttccacaagcggcacaagaacgcgctggcggaggcgctggagaagatcaaagcggagctgatgctggtggggttcatctcgctgctgctcgccgtgacgcaggacccaatctccgggatatgcatctccgagaaggccgccagcatcatgcggccctgcagcctgccccctggttccgtcaagagcaagtacaaagactactactgcgccaaaaaggtgagcctgctacaagctactcccggagacggccgggaaaaacacaaacagattccggcggccggccggagtttcttcttgtttccttcctgattggcttggcctaattggtgtgtgtttttctggcagggcaaggtgtcgctaatgtccacgggcagcttgcaccagctccacatattcatcttcgtgctcgccgtcttccatgtcacctacagcgtcatcatcatggctctaagccgtctcaaagtgagtctgtcaggcctacctgttcatgcttcggtaaagcaataaaactacttgctaccaatccctaatctgctccctcaggcataatattgttccttctttcctgctgcagatgaggacatggaagaaatgggagacagagaccgccgccacgaacttctctctgttaaagcaagcaggagacgtggaagaaaacccagggcccatggtgtccaagggcgaggagctcttcaccggcgtggtgccaatcctcgtggagctcgacggcgacgtgaacggccacaagttctccgtgtccggcgagggcgagggcgacgccacctacggcaagctcaccctcaagttcatctgcaccaccggcaagctcccagtgccatggccaaccctcgtgaccaccctctcccacggcgtgcaatgcttcgccaggtacccagaccacatgaagcaacacgacttcttcaagtccgccatgccagagggctacgtgcaagagaggaccatcttcttcaaggacgacggcaactacaagaccagggccgaggtgaagttcgagggcgacaccctcgtgaacaggatcgagctcaagggcatcgacttcaaggaggacggcaacatcctcggccacaagctcgagtacaacttcaactcccacaacgtgtacatcaccgccgacaagcaaaagaacggcatcaaggccaacttcaagatcaggcacaacatcgaggacggcggcgtgcaactcgccgaccactaccaacaaaacaccccaatcggcgacggcccagtgctcctcccagacaaccactacctctccacccaatccaagctctccaaggacccaaacgagaagagggaccacatggtgctcctcgagttcgtgaccgccgccggcatcaccctcggcatggacgagctctacaagtgaatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagtccttggaataccagttcgcaaatggtcagacaatttccgaaatgaaacctgactgatgcatttacaaacgcacgcaggcaggcatgatcagagtgagtgaactgatgatatgttttctctctctttcccgtgcctccagatcctgcgcggttccgcttcacgcaccagacgtcgttcgtgaagcgtcacctgggcctctccagcacccccggcatcagatgggtggtggccttcttcaggcagttcttcaggtcggtcaccaaggtggactacctcaccctgagggcaggcttcatcaacgtacgtaccaaaacaaatcctctccctctagcttcgccattgctgcgacgcttctgaaatatgtaccgttccgacaccagcgatctcatgtcttctctttccacgattctgcgcaggcgcatttgtcgcataacagcaagttcgacttccacaagtacatcaagaggtccatggaggacgacttcaaagtcgtcgttggcatcaggtaggttacattccatggataggattataaaattgccgtcaggctccatatgatattgcttaggttccacatgatacaatactatcagtttgctgcgtcatggtctttgcccctgctggtcttccttgcgtgatcttgaca

SEQ ID NO: 7 - DNA, 1837 bp, Artificial Sequence
  synthetically generated
aacagcgaggatgtccactacatgctcgaggccctggaagcccttggactctccgtggaagcagataaagttgcaaaaagagctgtggttgttggctgtggcggcaggttcccggtcgaaaaggacgccaaagaggaagtaaagctcttcttgggtaatgctggaattgcaatgcgaacattgacggcagctgtagtcgcagccggtggagccacgaacttctctctgttaaagcaagcaggagacgtggaagaaaacccagggcccatgcggtcttccaagaatgttatcaaggagttcatgaggtttaaggttcgcatggaaggaacggtcaatgggcacgagtttgaaatagaaggcgaaggagaggggaggccatacgaaggccacaataccgtaaagcttaaggtaaccaaggggggacctttgccatttgcttgggatattttgtcaccacaatttcagtatggaagcaaggtatatgtcaagcaccctgccgacataccagactataaaaagctgtcatttcctgaaggatttaaatgggaaagggtcatgaactttgaagacggtggcgtcgttactgtaacccaggattccagtttgcaggatggctgtttcatctacaaggtcaagttcattggcgtgaactttccttccgatggacctgttatgcaaaagaagacaatgggctgggaagccagcactgagcgtttgtatcctcgtgatggcgtgttgaaaggagagattcataaggctctgaagctgaaagacggtggtcattacctagttgaattcaaaagtatttacatggcaaagaagcctgtgcagctaccagggtactactatgttgactccaaactggatataacaagccacaacgaagactatacaatcgttgagcagtatgaaagaaccgagggacgccaccatctgttcctttaagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttaaatgcaacgtatgttttctttatcctagtgaaataagtatgagatccatgggtatgtttggagactgatcatgtcttttatttaaaaaaaaaacttcagttatgtgcttgatggagtaccaagaatgagggagcgacctattggtgacttggttgtcggtttgcaacaactcggcgcagatgctgattgtttccttggcactaactgcccacctgtccgtatcaatggcaaaggagggctacctggtggcaaggttagctactcatgaaattgcatgttatgtacttttgagcacattcagttctctgtcaaagaaaacaaatattatattggtagtacgatcaatgcgaccttacacaatgcactaaagttgaagaaccaataaacattgaataatttcaatacttgagtattgcatgatcaatcatagattgacacttaatattgaataatttcacaggttaagctctctggttccattagcagtcaatacctgagttccttgctgatggctgctcctttggctcttgaagatgtcgagattgaaatcattgataaactgatctccgttccttacgttgaaatgacattgaaattgatggagcgttttggcgtgactgcggcgcattctgatagttgggacaga

SEQ ID NO: 8 - DNA, 40 bp, Triticum aestivum
agtcccagggctggtgcaactggtggcccaggagggctgg

SEQ ID NO: 9 - DNA, 38 bp, Artificial Sequence
  synthetically generated
agtcccagggctggtgcaactggtgccaggagggctgg

SEQ ID NO: 10 - DNA, 38 bp, Artificial Sequence
  synthetically generated
agtcccagggctggtgcaactggtcccaggagggctgg

SEQ ID NO: 11 - DNA, 36 bp, Artificial Sequence
  synthetically generated
agtcccagggctggtgcaactgcccaggagggctgg

SEQ ID NO: 12 - DNA, 35 bp, Artificial Sequence
  synthetically generated
agtcccagggctggtgcaactggcaggagggctgg

SEQ ID NO: 13 - DNA, 34 bp, Artificial Sequence
  synthetically generated
agtcccagggctggtgcaactggtggagggctgg

SEQ ID NO: 14 - DNA, 34 bp, Artificial Sequence
  synthetically generated
agtcccagggctggtgcaactccaggagggctgg

SEQ ID NO: 15 - DNA, 93 bp, Artificial Sequence
  synthetically generated
acctgctgcatcaagcccccaagcttcaactactcctaccagtggccctgcaggtggtcttggaggcttatttccaggccttggtgctactgg

SEQ ID NO: 16 - DNA, 93 bp, Artificial Sequence
  synthetically generated
acctgctgcagcaagcccccaagcttcaaccactcctaccagtggccctgcaggtggtcttggaggcttatttccaggccttggtgctactgg

SEQ ID NO: 17 - DNA, 93 bp, Artificial Sequence
  synthetically generated
acctgctgcagcaagcccccaagcttcaactactcctaccagtggccctgcaggtggtcttggaggcttatttccaggccttggtgctactgg

SEQ ID NO: 18 - DNA, 82 bp, Artificial Sequence
  synthetically generated
ggcaatggcacaggtaattgttcttgattattatgttctttatgcacaatctcttcttccttgatatggattacgatgtcag

SEQ ID NO: 19 - DNA, 81 bp, Artificial Sequence
  synthetically generated
ggcaatggcacaggtattgttcttgattattatgttctttatgcacaatctcttcttccttgatatggattacgatgtcag

SEQ ID NO: 20 - DNA, 85 bp, Artificial Sequence
  synthetically generated
ggcaatggcacaggtaattgttcttgattattattatgttctttatgcacaatctcttcttccttgatatggattacgatgtcag

SEQ ID NO: 21 - DNA, 75 bp, Artificial Sequence
  synthetically generated
aagctcggccatgtaagttccctcactcctgcaacaagaaaaaaaaaagcctcaaccagaatcagcagctcagct

SEQ ID NO: 22 - DNA, 75 bp, Artificial Sequence
  synthetically generated
ctgctggtcttccttgcgtgatcttgacacatttggcctcttttcgcagcctcccgctgtggtgtgtggcgatcc

SEQ ID NO: 23 - DNA, 72 bp, Artificial Sequence
  synthetically generated
tgtagcttgtttctgctcattagggaacaacggtggtggataacctgttgaacagcgaggatgtccactaca

SEQ ID NO: 24 - DNA, 72 bp, Artificial Sequence
  synthetically generated
cgtagcttgtctctgctcattagggaacaacggtggtggataacctgttgaacagcgaggatgtccactaca

SEQ ID NO: 25 - DNA, 72 bp, Artificial Sequence
  synthetically generated
gcattctgatagttgggacagattctacatcaagggaggacaaaaatacaagtaagttctacattgctttac

SEQ ID NO: 26 - DNA, 72 bp, Artificial Sequence
  synthetically generate
gcattctgatagttgggacagattctacattaagggaggacaaaagtacaagtaagttctacattgctttac

SEQ ID NO: 27 - DNA, 20 bp, Artificial Sequence
  synthetically generated
gctggtgcaactggtggccc

SEQ ID NO: 28 - DNA, 20 bp, Artificial Sequence
  synthetically generated
gaactggtattccaaggagg

SEQ ID NO: 29 - DNA, 20 bp, Artificial Sequence
  synthetically generated
gtaatgctggaactgcaatg

SEQ ID NO: 30 - DNA, 20 bp, Artificial Sequence
  synthetically generated
gccacgaatgttccccactc

SEQ ID NO: 31 - DNA, 21 bp, Artificial Sequence
  synthetically generated
aaaaaggagaacacatgcaca

SEQ ID NO: 32 - DNA, 22 bp, Artificial Sequence
  synthetically generated
gggacaactccagtgaaaagtt

SEQ ID NO: 33 - DNA, 20 bp, Artificial Sequence
  synthetically generated
acggaacctgggtgcagatg

SEQ ID NO: 34 - DNA, 20 bp, Artificial Sequence
  synthetically generated
ccccgtcgtggtccttgtag

SEQ ID NO: 35 - DNA, 20 bp, Artificial Sequence
  synthetically generated
gctgccaaagactgcaacca

SEQ ID NO: 36 - DNA, 20 bp, Artificial Sequence
  synthetically generated
gccacgaatgttccccactc

SEQ ID NO: 37 - DNA, 20 bp, Artificial Sequence
  synthetically generated
atcctcggccacaagttgga

SEQ ID NO: 38 - DNA, 20 bp, Artificial Sequence
  synthetically generated
gtggcgggtcttgaagttgg

SEQ ID NO: 39 - DNA, 20 bp, Artificial Sequence
  synthetically generated
tgcagtgcttcagccgctac

SEQ ID NO: 40 - DNA, 20 bp, Artificial Sequence
  synthetically generated
tcgccctcgaacttcacctc

SEQ ID NO: 41 - DNA, 20 bp, Artificial Sequence
  synthetically generated
gctggaaacggctaggagca

SEQ ID NO: 42 - DNA, 20 bp, Artificial Sequence
  synthetically generated
tggctggaacagcacctcag

SEQ ID NO: 43 - DNA, 20 bp, Artificial Sequence
  synthetically generated
ttgagcaactcatggaccag

SEQ ID NO: 44 - DNA, 20 bp, Artificial Sequence
  synthetically generated
gctttccaaggcacaaacat

SEQ ID NO: 45 - DNA, 17 bp, Artificial Sequence
  synthetically generated
ccaaacccctggagcaa

SEQ ID NO: 46 - DNA, 21 bp, Artificial Sequence
  synthetically generated
ggttcatgctaagcaactgtg

SEQ ID NO: 47 - DNA, 28 bp, Artificial Sequence
  synthetically generated
gcttcaactactcctaccagtggccctg

SEQ ID NO: 48 - DNA, 28 bp, Artificial Sequence
  synthetically generated
gcttgccggtggtgcagatgaacttcag

SEQ ID NO: 49 - DNA, 30 bp, Artificial Sequence
  synthetically generated
cctgttgccggtcttgcgatgattatcata

SEQ ID NO: 50 - DNA, 30 bp, Artificial Sequence
  synthetically generated
gagaactctggagcattcacatcaaacctg

SEQ ID NO: 51 - DNA, 20 bp, Artificial Sequence
  synthetically generated
acaagctcggccatgtaagt

SEQ ID NO: 52 - DNA, 24 bp, Artificial Sequence
  synthetically generated
tgctttaacagagagaagttcgtg

SEQ ID NO: 53 - DNA, 26 bp, Artificial Sequence
  synthetically generated
aagtgaatatgaagatgaagatgaaa

SEQ ID NO: 54 - DNA, 20 bp, Artificial Sequence
  synthetically generated
tgaacggtggttacgagaca

SEQ ID NO: 55 - DNA, 22 bp, Artificial Sequence
  synthetically generated
tttcttttctgatggacccttt

SEQ ID NO: 56 - DNA, 24 bp, Artificial Sequence
  synthetically generated
tgctttaacagagagaagttcgtg

SEQ ID NO: 57 - DNA, 20 bp, Artificial Sequence
  synthetically generated
agcgcgcaaactaggataaa

SEQ ID NO: 58 - DNA, 27 bp, Artificial Sequence
  synthetically generated
tatcagaagaagtaaagcaatgtagaa

(19) United States
(12) Patent Application Publication
(10) Pub. No.: US 2019/0249183 A1
(43) Pub. Date: Aug. 15, 2019

(54) MULTIPLEX GENE TARGETING IN PLANTS

(71) Applicants: Javier Gil Humanes, Circle Pines, MN (US); Daniel F. Voytas, Falcon Heights, MN (US); Nicholas J. Baltes, Maple Grove, MN (US)

(72) Inventors: Javier Gil Humanes, Circle Pines, MN (US); Daniel F. Voytas, Falcon Heights, MN (US); Nicholas J. Baltes, Maple Grove, MN (US)

(21) Appl. No.: 16/344,496

(22) PCT Filed: Oct. 24, 2017
(86) PCT No.: PCT/US2017/058031

§ 371 Date: Apr. 24, 2019

Related U.S. Application Data
(60) Provisional application No. 62/411,952, filed on Oct. 24, 2016.

(51) Int. Cl. C12N 15/82; C12N 15/11; C12N 9/22

(52) U.S. Cl. CPC C12N 15/8213; C12N 15/11; C12N 2800/80; C12N 2310/20; C12N 9/22

(57) ABSTRACT

Materials and methods for high efficiency gene targeting at multiple genomic sites in a single cell are provided herein.

### CROSS-REFERENCE TO RELATED APPLICATIONS

This application claims benefit of priority from U.S. Provisional Application Ser. No. 62/411,952, filed on Oct. 24, 2016, which is incorporated herein by reference in its entirety.

### STATEMENT AS TO FEDERALLY SPONSORED RESEARCH

This invention was made with government support under IOS-1339209 awarded by the National Science Foundation. The government has certain rights in the invention.

### TECHNICAL FIELD

This document relates to materials and methods for high efficiency gene targeting at multiple genomic sites in a single cell.

### BACKGROUND

The ability to edit plant genomes through gene targeting (GT) has traditionally been hindered by low frequencies of recombination. Editing plant genomes through GT requires efficient methods to deliver both sequence-specific nucleases (SSNs) and repair templates to plant cells. This can be achieved using Agrobacterium T-DNA or biolistics, or by stably integrating nuclease-encoding cassettes and repair templates into the plant genome. In dicotyledonous plants such as tobacco and tomato, for example, greater than 10-fold enhancements in GT frequencies have been achieved using DNA virus-based replicons. These replicons transiently amplify to high copy numbers in plant cells, delivering abundant SSNs and repair templates to achieve targeted gene modification. While the use of SSNs has helped to increase recombination frequencies, successes in GT have largely been limited to single-gene, single-loci targets.

### SUMMARY

This document is based on the discovery of materials and methods that enable multiplex gene targeting in plants. For example, this document is based, at least in part, on the development of a replicon-based system for genome engineering of plants (e.g., cereal crops) using a deconstructed version of the wheat dwarf virus (WDV). As described herein, the replicons achieved more than a 100-fold (e.g., about 110-fold) increase in expression of a reporter gene in wheat cells, relative to non-replicating controls. Replicons carrying CRISPR/Cas9 nucleases and repair templates achieved GT at an endogenous ubiquitin locus at frequencies at least 10 fold (e.g., about 12-fold) greater than non-viral delivery methods. Moreover, in some cases, the methods provided herein with the deconstructed WDV replicons can be used for gene targeted integration by HR in all three homeoalleles (A, B, and D) of the hexaploid wheat genome, thus achieving multiplexed GT within the same wheat cell. Thus, these materials and methods can provide high frequencies of GT that make it possible to edit complex genomes without the need to integrate GT reagents into the genome.

In one aspect, this document features a method for modifying genomic material of a plant cell at two or more loci. The method can include (a) providing a plant cell that contains two or more endogenous nucleic acid sequences to be modified, (b) introducing into the plant cell (i) a first repair template targeted to a first genomic sequence within the plant cell, and (ii) a second repair template targeted to a second genomic sequence within the plant cell, and (c) maintaining the plant cell under conditions in which the first and second repair templates recombine by homologous recombination with their corresponding genomic loci, thereby producing a plant cell containing targeted genomic modifications at the first and second genomic sequences.

The first repair template can be within a first geminivirus replicon that includes, in order from 5′ to 3′, a first geminivirus long intergenic region (LIR), the first repair template, a geminivirus short intergenic region (SIR), a virus Rep/RepA coding sequence, and a second geminivirus LIR. The second repair template can be within a second geminivirus replicon that contains, in order from 5′ to 3′, a third geminivirus LIR, the second repair template, a second geminivirus SIR, a virus Rep/RepA coding sequence, and a fourth geminivirus LIR. The first, second, third, and fourth geminivirus LIRs can contain the same nucleotide sequence, or at least two of the first, second, third, and fourth geminivirus LIRs can contain different nucleotide sequences. The first and second geminivirus SIRs can contain the same nucleotide sequence, or can contain different nucleotide sequences. The first and second repair templates can be within a geminivirus replicon that contains, in order from 5′ to 3′, a first geminivirus LIR, the first repair template, the second repair template, a geminivirus SIR, a virus Rep/RepA coding sequence, and a second geminivirus LIR. The first and second geminivirus LIRs can contain the same nucleotide sequence, or can contain different nucleotide sequences.

In some embodiments, the method can further include introducing into the plant cell a first sequence specific endonuclease targeted to the first genomic sequence and a second sequence specific endonuclease targeted to the second genomic sequence, and maintaining the plant cell under conditions in which the first and second sequence specific endonucleases are expressed to introduce double stranded DNA breaks (DSBs) at the first and second genomic sequences. The first and second sequence specific endonucleases can be first and second Cas9 endonucleases, and the method can further include introducing a first guide RNA that targets the first Cas9 endonuclease to the first genomic sequence, and a second guide RNA that targets the second Cas9 endonuclease to the second genomic sequence.

The geminivirus can be, for example, wheat dwarf virus or bean yellow dwarf virus. The plant cell is from a polyploid plant (e.g., wheat, oat, triticale, tritordeum, peanut, sugar cane, white potato, tobacco, apple, banana, watermelon, canola, leek, strawberry, or cotton). The plant cell can be a protoplast. All alleles and homeoalleles of the two or more endogenous nucleic acid sequences may be modified in the plant cell containing the targeted genomic modifications. The method can include introducing the first and second repair templates into the plant cell simultaneously, or introducing the first and second repair templates into the plant cell sequentially.

The method can further include introducing into the plant cell a third repair template targeted to a third genomic sequence within the plant cell, and maintaining the plant cell under conditions in which the third template recombines by homologous recombination with its corresponding genomic sequence. The third repair template can be within a third geminivirus replicon that contains, in order from 5′ to 3′, a fifth geminivirus LIR, the third repair template, a third geminivirus SIR, a virus Rep/RepA coding sequence, and a sixth geminivirus LIR. The first, second, and third repair templates can be within a geminivirus replicon that contains, in order from 5′ to 3′, a first geminivirus LIR, the first repair template, the second repair template, the third repair template, a first geminivirus SIR, a virus Rep/RepA coding sequence, and a second geminivirus LIR. The method can further include introducing into the plant cell a first sequence specific endonuclease targeted to the first genomic sequence, a second sequence specific endonuclease targeted to the second genomic sequence, and a third sequence specific endonuclease targeted to the third genomic sequence, and maintaining the plant cell under conditions under which the first, second, and third sequence specific endonucleases are expressed to introduce DSBs at the first, second, and third genomic sequences. The first, second, and third sequence specific endonucleases can be first, second, and third Cas9 endonucleases, and the method can further include introducing a first guide RNA that targets the first Cas9 endonuclease to the first genomic sequence, a second guide RNA that targets the second Cas9 endonuclease to the second genomic sequence, and a third guide RNA that targets the third Cas9 endonuclease to the third genomic sequence. The method can include introducing the first, second, and third repair templates into the plant cell simultaneously, or introducing the first, second, and third repair templates into the plant cell sequentially.

In another aspect, this document features a nucleic acid containing a first sequence that includes, in order from 5′ to 3′, a first geminivirus LIR, a first repair template targeted to a first genomic sequence within a first of two or more endogenous plant nucleic acid sequences, a geminivirus SIR, a virus Rep/RepA coding sequence, and a second geminivirus LIR. The geminivirus can be wheat dwarf virus or bean yellow dwarf virus. The nucleic acid can further include a sequence encoding a first sequence specific endonuclease that targets the first endogenous plant nucleic acid sequence. The sequence specific endonuclease can be a Cas9 endonuclease, and the nucleic acid can further include a sequence encoding a first guide RNA that targets the first Cas9 endonuclease to the first genomic sequence. The nucleic acid can further contain a second sequence that contains, in order from 5′ to 3′, a third geminivirus LIR, a second repair template targeted to a second endogenous nucleic acid sequence within the plant, a second geminivirus SIR, a virus Rep/RepA coding sequence, and a fourth geminivirus LIR. The first, second, third, and fourth geminivirus LIRs can contain the same nucleotide sequence, or at least two of the first, second, third, and fourth geminivirus LIRs can contain different nucleotide sequences. The first and second geminivirus SIRs can contain the same nucleotide sequence, or can contain different nucleotide sequences.

In some embodiments, the nucleic acid can contain, in order from 5′ to 3′, the first geminivirus LIR, the first repair template, a second repair template targeted to a second endogenous nucleic acid sequence within the plant, the geminivirus SIR, the virus Rep/RepA coding sequence, and the second geminivirus LIR. The nucleic acid can further contain a sequence encoding a first sequence specific endonuclease that targets the first endogenous plant nucleic acid sequence, and a sequence encoding a second sequence specific endonuclease that targets the second endogenous plant nucleic acid sequence. The first and second sequence-specific endonucleases can be first and second Cas9 endonucleases, and the nucleic acid can further contain a sequence encoding a first guide RNA that targets the first Cas9 endonuclease to the first genomic sequence, and a sequence encoding a second guide RNA that targets the second Cas9 endonuclease to the second genomic sequence. The first and second geminivirus LIRs can contain the same nucleotide sequence, or can contain different nucleotide sequences. The plant can be a polyploid plant (e.g., wheat, oat, triticale, tritordeum, peanut, sugar cane, white potato, tobacco, apple, banana, watermelon, canola, leek, strawberry, or cotton).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram of the replication cycle of WDV-derived replicons in transformed plant cells. For genome engineering purposes, a WDV-derived replicon is delivered into plant cell nuclei by particle bombardment (double-stranded DNA) or Agrobacterium-mediated transformation (single-stranded T-DNA). A ssDNA replicon is then released and converted into dsDNA by host polymerases. The replication initiation protein (Rep) recognizes a domain in the large intergenic region (LIR) and nicks the DNA at a 9-nt conserved site found on the hairpin structure of the LIR to promote rolling circle replication (RCR). As a result, newly synthesized ssDNA replicons are formed and converted again into dsDNA replicons. The new dsDNA replicons are used to either express the encoded proteins [e.g., Rep, RepA, and heterologous proteins such as green fluorescent protein (GFP)] or start a new RCR cycle.

FIG. 2 is a schematic showing different exemplary architectures of WDV replicons. WDV1: the maize Ubiquitin 1 (ZmUbi) promoter drives the expression of the heterologous sequence. WDV2: the LIR drives expression of the heterologous sequence. WDV3: contains a premature stop codon in the Rep/RepA coding sequence, and consequently replication is impaired.

FIGS. 3A-3C are images showing GFP expression mediated by different geminiviruses in wheat calli. Bright field photos are shown in the left panels, and GFP photos are shown in the right panels. FIG. 3A, Bean Yellow Dwarf Virus; FIG. 3B, Tomato Leaf Curl Virus; and FIG. 3C, Wheat Dwarf Virus.

FIGS. 4A-4C are images showing GFP expression and circularization of WDV vectors in transfected rice protoplasts for pWDV2-GFP (FIG. 4A), pWDV3-GFP (FIG. 4B), and Ubi-GFP ctrl (FIG. 4C). Arrows denote specific primers used to detect circularization of the WDV replicon in FIG. 4A and FIG. 4B.

FIG. 5 shows the complete sequence of wheat codon-optimized Cas9 (TaCas9; SEQ ID NO:1). The MASS translation initiation signal is indicated by italics, the 3× Flag tag sequence is bold, and the two nuclear localization signals (NLS) are underlined.

FIG. 6A shows the complete sequence of the sgRNA expression cassette (SEQ ID NO:2). The polymerase III promoter U6 from wheat (TaU6) is shown in bold, and the sgRNA Esp3I cloning site is shown in italics with the Esp3I sites underlined. FIG. 6B shows the sgRNA sequence, which is composed of two oligonucleotides (SEQ ID NOS:3 and 4) with overhang ends (italics) flanking the protospacer sequence (G-N19), where the G is needed for expression of the sgRNA by the U6 promoter, and represents position 1 of the gRNA.

FIG. 7A is a schematic of the T2A:gfp donor template. FIG. 7B shows the complete sequence of the T2A:gfp donor template (SEQ ID NO:5), indicating the location of the left and right homology arms (italics), the self-cleavage peptide T2A (bold), the gfp, coding sequence (bold and underlined), and the nos terminator (underlined). The target sequence of the sgUbi1 is shown in bold italics, with the downstream mutated PAM sequence (ACC) underlined and in italics.

FIG. 8A is schematic of the P2A:bfp donor template. FIG. 8B shows the complete sequence of the P2A:bfp donor template (SEQ ID NO:6), indicating the location of the left and right homology arms (italics), the self-cleavage peptide P2A (bold), the bfp coding sequence (bold, underlined), and the HSP terminator (underlined). The target sequence of the sgMLO1 is shown in bold italics, with the downstream PAM sequence (CGG) underlined. The homologous sequence to the sgMLO1 recognizes the complement strand and is split into the two homology arms to avoid cleavage of the donor template by TaCas9.

FIG. 9A is a schematic of the P2A:dsRed donor template. FIG. 9B shows the complete sequence of the P2A:dsRed donor template (SEQ ID NO:7), indicating the locations of the left and right homology arms (italics), the self-cleavage peptide P2A (bold), the dsRed coding sequence (bold, underlined), and the nos terminator (underlined). The target sequence of the sgUbi1 is shown in bold italics, with the downstream mutated PAM sequence (CGA) underlined.

FIGS. 10A-10E show GFP expression and circularization of the WDV vectors in transformed corn calli using pWDV1-GFP (FIG. 10A), pWDV2-GFP (FIG. 10B), pWDV3-GFP (FIG. 10C), and Ubi-GFP ctrl (FIG. 10D). Arrows in FIG. 10E denote specific primers used to detect circularization of the WDV replicon.

FIGS. 11A-11E are a series of graphs and images showing WDV replicon replication and performance of different WDV architectures designed for genome engineering of cereal species. FIG. 11A is a graph plotting the time course of WDV replication, as well as expression of GFP and Rep/RepA proteins in wheat calli at different days post bombardment (dpb). FIG. 11B is a graph plotting the normalized copy number of the GFP-containing replicon relative to the Ubi-GFP control at 5 dpb. FIG. 11C is a series of images showing GFP expression in wheat calli using the indicated WDV architectures: WDV1-GFP (left panel), WDV2-GFP (central panel), and WDV3-GFP (right panel). FIG. 11D is a graph plotting qRT-PCR quantification of gene expression normalized to the non-replicating pWDV3-GFP. FIG. 11E is a graph plotting replicon copy number normalized to the non-replicating pWDV3-GFP (top), and an image indicating detection of circularized replicon and actin PCR control (bottom). Arrows in FIG. 11E indicate primers used to detect circularization in each replicon variant. Error bars represent the standard error (SE) of three independent biological replicates (n=6 wheat calli per replicate) transformed by particle bombardment. Gold particles with no DNA were used to transform the wild type (WT) control.

FIGS. 12A-12C show WDV replicon-mediated expression of the CRISPR/Cas9 system for targeted mutagenesis in wheat cells. FIG. 12A is a diagram depicting CRISPR/Cas reagents expressed from the different architectures of the WDV replicons. FIG. 12B shows the results of a PCR/restriction enzyme assay to detect mutations in the ubiquitin gene induced with the sgUbi1 expressed in the different vectors shown in FIG. 12A. *=Percentages of NHEJ (%±SE) representing the average of two different transfections, normalized to the transfection efficiency (40% and 60%, respectively). FIG. 12C shows a wild type ubiquitin sequence (SEQ ID NO:8) and nucleotide sequences (SEQ ID NOS:9-14) from resistant bands obtained by NHEJ of the ubiquitin gene mediated by the pWDV1-CR vector. The protospacer sequence is underlined, with the PAM sequence in bold and italics.

FIGS. 13A-13D demonstrate high efficiency GT mediated by WDV-derived replicons expressing CRISPR/Cas9 reagents in wheat cells. FIG. 13A is a series of images showing GT-mediated expression of GFP in wheat protoplasts two days after transfection with the indicated WDV vectors carrying the CRISPR/Cas9 reagents and the T2A:gfp donor template. Quantification of the number of GFP positive cells was carried out by flow cytometry (lower panels). FIG. 13B is a schematic showing the expected integration of the T2A:gfp sequence in the genomic ubiquitin locus (top), and images of a gel for molecular characterization by PCR using specific primers. Lane numbers in the gel denote the following constructs: 1, pWDV1.CR.GFP; 2, pWDV2.CR.GFP; 3, pWDV3.CR.GFP. FIG. 13C shows DNA sequences for the 5′ (top; SEQ ID NOS:15, 16, and 17) and 3′ (bottom, SEQ ID NOS:18, 19, and 20) junctions of the integrated T2A:gfp fragment obtained with the pWDV1.CR.GFP vector. FIG. 13D includes a graph and an image of a gel showing enhancement of GT efficiency in wheat scutella mediated by the WDV-derived replicons. Black bars denote normalized GT efficiency compared to the non-viral control (pCR.GFP, hatched bar). Error bars represent the standard error (SE) of five different transformation experiments (n=132 scutella for each treatment). Replicon circularization and the endogenous actin gene control were detected by PCR. Total GT frequency (%±SE) represents the percentage of cells with GT events relative to the total number of transformed cells (as calculated with the pWDV1-GFP control in each individual experiment). The average percentage of scutella showing at least one GT event is shown in parenthesis.

FIG. 14 is a series of images showing GT of the T2A:gfp sequence into wheat scutella. Arrows indicate GFP-expressing cells in wheat scutella 7 dpb.

FIGS. 15A-15E demonstrate single-cell multiplexed GT mediated using the WDV-CRISPR/Cas9 system in wheat cells. FIG. 15A is an image showing GT-mediated expression of BFP in wheat protoplasts two days after transfection with WDV vectors carrying the CRISPR/Cas9 reagents and the P2A:bfp donor template. Quantification of BFP positive cells was carried out by image quantification. FIG. 15B is a schematic showing the expected integration of the P2A:bfp sequence in the genomic MLO locus (top), and PCR amplification of the 5′ and 3′ junctions using specific primers (bottom). FIG. 15C shows DNA sequences (SEQ ID NOS:20 and 21) for the 5′ and 3′ junctions, indicating knock-in of the P2A:bfp into the MLO homeoallele in the D genome. FIG. 15D is a series of images indicating multiplexed GT of the promoter-less T2A:gfp and P2A:bfp sequences in wheat protoplasts. GFP and BFP channels are shown in the left and center images, respectively. The right image is a merge of the two single channel images; the arrow denotes a cell expressing both GFP and BFP. FIG. 15E is a diagram of the GFP, BFP, and GFP+BFP knock-in frequencies relative to the total number of cells that had undergone GT.

FIGS. 16A-16D show single-cell multiplexed GT of GFP and dsRed mediated by the WDV-CRISPR/Cas9 system in wheat scutella. FIG. 16A is a series of images showing multiplexed GT of the promoter-less T2A:gfp and P2A:dsRed sequences into two different loci in wheat scutella. dsRed and GFP channels are shown in the left and center images, respectively, for two scutella transformed with pWDV1.CR.GFP+dsRed (diagram at top). The images on the right represent a merge of the two single channel images for each scutellum. Arrows denote single cells expressing dsRed fluorescence and single cells expressing GFP fluorescence (left panels, center panels, and top right panel). The arrow in the bottom right panel indicates a single cell expressing both dsRed and GFP fluorescence. FIG. 16B is a diagram indicating the frequency of cells showing GT of the GFP (large circle), dsRed (small circle), and both GFP and dsRed (white intersection). FIG. 16C is a schematic (top) and an image of a gel (bottom) indicating integration of the P2A:dsRed sequence in the EPSPS locus. FIG. 16D shows DNA sequences for the 5′ (SEQ ID NOS:23 and 24) and 3′ (SEQ ID NOS:25 and 26) junctions, indicating knock-in of P2A:dsRed into the EPSPS homeoalleles of the A and D genomes.

### DETAILED DESCRIPTION

Methods to precisely edit cellular genomes can utilize repair templates, optionally in combination with highly efficient and programmable SSNs, including meganucleases (Puchta et al., Nucleic Acids Res 1993, 21:5034-5040; Salomon and Puchta, EMBO J 1998, 17:6086-6095; and Jacoby et al., Nucl. Acids Res., 10.1093/nar/gkr1303, 2012), zinc-finger nucleases (ZFNs) (Kim et al., Proc Natl Acad Sci USA 1996, 93:1156-1160; Townsend et al., Nature 2009, 459:442-445; and Sander et al., Nature Methods, 8:67-69, 2011), transcription activator-like effector (TALE) nucleases (Christian et al., Genetics 2010, 186:757-761; Bogdanove and Voytas, Science 2011, 333:1843-1846; and U.S. Publication No. 2011/0145940), and the clustered regularly interspaced short palindromic repeat (CRISPR)-Cas9 system (Hwang et al., Nat Biotechnol 2013, 31:227-229; Shan et al., Nat Biotechnol 2013, 31:686-688; Cong et al., Science 339:819-823, 2013; and Mali et al., Science 339:823-826, 2013). SSNs can be used to introduce a double-strand break (DSB) in the target locus to be modified, and the DSB can be repaired by one of the two primary pathways: non-homologous end joining (NHEJ) or homologous recombination (HR). In NHEJ, the ends of the broken chromosome are rejoined, sometimes imprecisely, which can introduce small insertions or deletions (indels) at the break site (Gorbunova and Levy, Nucleic Acids Res 1997, 25:4650-4657). When indels occur in coding sequences, they may create frame shift mutations that disrupt gene function. In HR, or gene targeting (GT), the DSB is repaired using a template with homology to the break site. The repair template can be the sister chromatid, a homologous or homeologous chromosome (in the case of polyploid species), or an exogenous template containing one or more specific sequence modifications to be incorporated into the break site. Efficient delivery of genome engineering reagents to plant cells is necessary to achieve targeted genome modification, particularly GT, since GT involves delivery of both a SSN expression cassette and a repair template.

This document provides materials and methods for efficient delivery of genome engineering reagents that can be used to achieve GT in plants, including crop species such as cereals that are difficult to transform. In some embodiments, the methods provided herein can utilize DNA viruses, such as geminiviruses, that have been engineered as vectors for the expression of heterologous proteins in plants. Although the cargo capacity of these viruses is not unlimited, they can be converted into non-infectious replicons by replacing genes important for infection and cell-to-cell movement with heterologous sequences (Lazarowitz et al., supra; Shen and Hohn The Plant Journal 1994, 5:227-236; and Shen and Hohn J Gen Virol 1995, 76 (Pt 4):965-969), including SSN expression cassettes and repair templates (Lazarowitz et al., EMBO J 1989, 8:1023-1032; and Ugaki et al., Nucleic Acids Res 1991, 19:371-377).

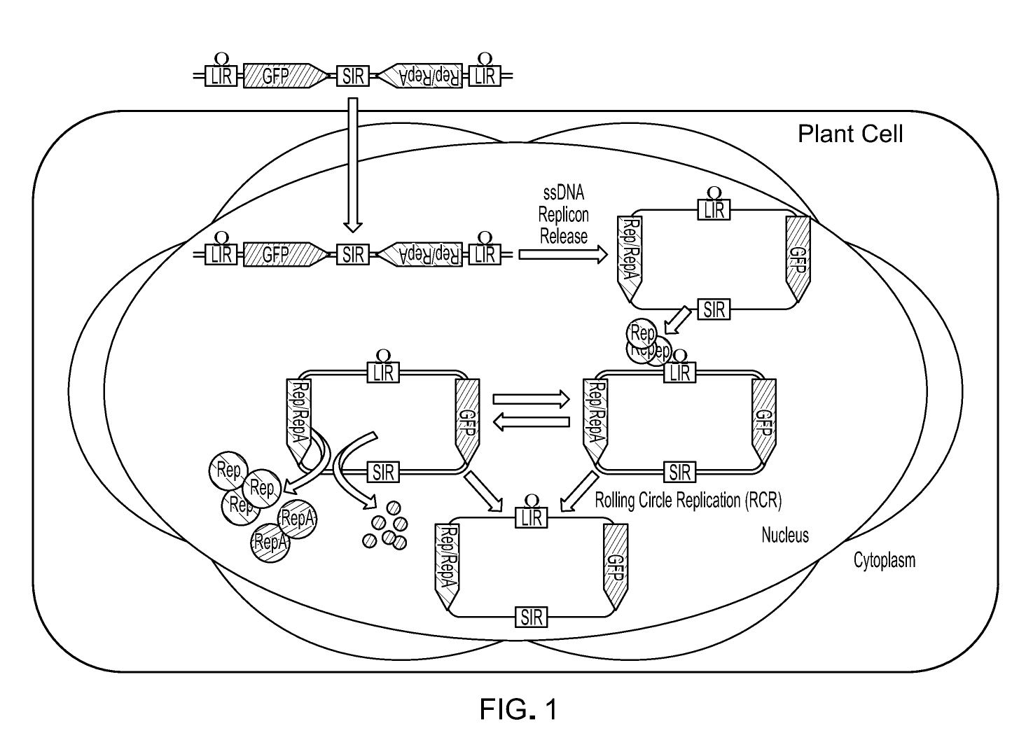

Geminiviruses replicate through a rolling circle replication (RCR) cycle (FIG. 1), and consequently, viral replicons can achieve high copy number, increasing the transient expression of the SSN and repair template. For example, a deconstructed version of bean yellow dwarf virus (BeYDV), was used to deliver ZFNs and a repair template to tobacco cells to achieve gene targeting at an integrated reporter gene (Baltes et al., Plant Cell 2014, 26:151-163); BeYDV also have been used for targeted knock-in of a strong promoter upstream of a tomato gene that regulates anthocyanin synthesis (Čermák et al., Genome Biol 16:232, 2015). WDV is a ssDNA virus (Mastrevirus) that infects a variety of grasses, including most cereals. WDV-derived replicons can be used to express foreign proteins in cells from plants such as wheat and maize cells (Ugaki et al., supra; Matzeit et al., Plant Cell 1991, 3:247-258; and Suarez-Lopez and Gutierrez, Virology 1997, 227:389-399). Tomato leaf curl virus (ToLCV) also is a ssDNA virus (Begomovirus), and although its natural hosts are normally Solanaceous species, ToLCV-derived replicons can efficiently replicate and express GFP in rice (Pandey et al., Virol J 2009, 6:152).

The replicon-based systems described herein that are useful as GT vectors have characteristics that may include one or more of the following: (a) the viral DNA genome can be used as repair template, (b) the replicon (including the repair template) can replicate to high copy number, and (c) the expression of Rep and RepA viral proteins can enhance HR, perhaps due to the interaction with proteins in the plant cell that promote progression into S phase.

As described herein, replicons based on WDV were developed for plant genome engineering. The WDV-derived replicons were successfully used to amplify and express heterologous proteins in plants such as wheat, corn, and rice. The Example section herein describes the replication and protein expression of the WDV system in wheat cells, and provides different replicon architectures that were used to optimize WDV as a vector for delivering CRISPR/Cas reagents and donor templates. Use of the WDV replicons increased GT efficiency greater than 10-fold in wheat cells. In addition, the replicons were able to promote multiplexed GT, achieving targeted integration, within the same cell, of different reporter genes in different loci of the polyploid wheat genome.

This document provides highly efficient, virus-based systems and methods for targeted modification of plant genomes. The in planta systems and methods for GT include the use of customizable endonucleases in combination with plant DNA virus-based replicons. Plant DNA viruses, including geminiviruses, have many attributes that may be advantageous for in planta GT, including their ability to replicate to high copy numbers in plant cell nuclei. These viruses can be modified to encode a desired nucleotide sequence, such as a repair template sequence targeted to a particular sequence in a plant genome. First generation geminiviruses, or “full viruses” (viruses that retain only the useful “blocks” of sequence), can carry up to about 800 nucleotides (nt), while deconstructed geminiviruses (viruses that encode only the proteins needed for viral replication) have a much larger cargo capacity. This document describes how customizable nucleases and plant DNA viruses can be used for in planta GT, and provides materials and methods for achieving such GT. The methods can be used with both monocotyledonous plants [e.g., banana, grasses (such as Brachypodium distachyon), wheat, oats, barley, maize, Haynaldia villosa, palms, orchids, onions, pineapple, rice, and sorghum] and dicotyledonous plants (e.g., Arabidopsis, beans, Brassica, carnations, chrysanthemums, citrus plants, coffee, cotton, eucalyptus, impatiens, melons, peas, peppers, Petunia, poplars, potatoes, roses, soybeans, squash, strawberry, sugar beets, tobacco, tomatoes, and woody tree species), and can be particularly useful with plants having complex, polyploid genomes [e.g., durum wheat (Triticum turgidum durum), which is tetraploid, white bread wheat (Triticum aestivum), which is hexaploid, as well as other triploid species (e.g., apple, banana, and watermelon), tetraploid species (e.g., cotton, potato, canola, leek, tobacco, and peanut), hexaploid species (e.g., oat, triticale, and tritordeum), and octoploid species (e.g., strawberry and sugar cane)].

In general, the systems and methods described herein include two components: a plant DNA virus-based replicon containing a repair template targeted to an endogenous plant sequence, and an endonuclease that also is targeted to a site near or within the target sequence. The endonuclease can generate a targeted DNA DSB at the desired locus, and the plant cell can repair the DSB using the repair template present in the replicon, thereby incorporating the modification stably into the plant genome. In some embodiments, the systems and methods provided herein include two or more plant DNA virus-based replicons that each contain a different repair template with or without a sequence encoding a SSN, or include a plant DNA virus-based replicon that contains two or more repair templates targeted to endogenous plant sequences, with or without sequences encoding two or more SSNs that also are targeted to sites near or within the target sequences. The endonucleases can generate targeted DNA DSBs at the desired loci, and the plant cell can repair the DSBs using the repair templates present in the replicon(s), thereby incorporating the modifications stably into the plant genome.

Geminivirus-based replicons can be particularly useful. Geminiviruses are a large family of plant viruses that contain circular, single-stranded DNA genomes. Examples of geminiviruses include the cabbage leaf curl virus, tomato golden mosaic virus, bean yellow dwarf virus (BeYDV; also referred to as chickpea chlorotic dwarf virus), African cassava mosaic virus, wheat dwarf virus (WDV), miscanthus streak mastrevirus, tobacco yellow dwarf virus, tomato yellow leaf curl virus, bean golden mosaic virus, beet curly top virus, maize streak virus, and tomato pseudo-curly top virus.

In some embodiments, a first component of the systems and methods described herein is a geminivirus-based replicon engineered to contain a repair template that includes a desired modification (a “donor sequence”) that is heterologous to the plant to be modified, flanked by sequences of homology (“homology arms”) to a target locus within the plant genome. The engineered replicon can be generated by, for example, replacing non-essential geminivirus nucleotide sequence (e.g., CP sequence) with a desired repair template. Other methods for adding sequence to viral vectors include, without limitation, those discussed in Peretz et al. (Plant Physiol., 145:1251-1263, 2007).

A repair template as used herein can include a donor nucleic acid sequence having the ability to replace an endogenous target sequence within the plant, flanked by homology arms containing sequences homologous to endogenous sequences on either side of the target. A repair template can have a length ranging from about 25-50 nucleotides up to about 10,000 nt (e.g., about 25 nt to about 50 nt, about 50 nt to about 100 nt, about 100 nt to about 300 nt, about 300 nt to about 500 nt, about 500 nt to about 700 nt, about 700 nt to about 1000 nt, about 1000 nt to about 1500 nt, about 1500 nt to about 2000 nt, about 2500 nt to about 3000 nt, about 3000 nt to about 5000 nt, about 5000 nt to about 7500 nt, or about 7500 nt to about 10,000 nt). Within a repair template, each homology arm can have a length of about 50 nt to about 5000 nt (e.g., about 50 nt to about 100 nt, about 100 nt to about 300 nt, about 300 nt to about 500 nt, about 500 nt to about 700 nt, about 700 nt to about 1000 nt, about 1000 nt to about 3000 nt, or about 3000 nt to about 5000 nt). The donor sequence between the homology arms can have a length from about 1 nt to about 5000 nt (e.g., about 1 nt to about 50 nt, about 50 nt to about 100 nt, about 100 nt to about 200 nt, about 200 nt to about 300 nt, about 300 nt to about 400 nt, about 400 nt to about 500 nt, about 500 nt to about 1000 nt, about 1000 nt to about 2000 nt, about 2000 nt to about 3000 nt, or about 3000 nt to about 5000 nt). Repair templates and DNA virus plasmids can be prepared using molecular biology techniques such as those that are described in the Example section.

The homology arms within a repair template can have at least about 90% sequence identity (e.g., at least about 90%, 92%, 94%, 95%, 96%, 97%, 98%, 99%, or 99.5% sequence identity) to the endogenous plant sequences to which they are targeted.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -l -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:2), or by an articulated length (e.g., 100 consecutive nucleotides or amino acid residues from a sequence set forth in an identified sequence), followed by multiplying the resulting value by 100. For example, a nucleic acid sequence that has 450 matches when aligned with the sequence set forth in SEQ ID NO:2 is 96.4 percent identical to the sequence set forth in SEQ ID NO:2 (i.e., 450÷467×100=96.4). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

A second component of the systems and methods described herein can be an endonuclease that can be customized to target a particular nucleotide sequence and generate a DSB at or near that sequence. As noted above, examples of customizable endonucleases include ZFNs, meganucleases, and TALE nucleases, as well as CRISPR/Cas systems. In particular, CRISPR/Cas molecules are components of a prokaryotic adaptive immune system that is functionally analogous to eukaryotic RNA interference, using RNA base pairing to direct DNA or RNA cleavage. Directing DNA DSBs requires two components: the Cas9 protein, which functions as an endonuclease, and CRISPR RNA (crRNA) and tracer RNA (tracrRNA) sequences that aid in directing the Cas9/RNA complex to target DNA sequence (Makarova et al., Nat Rev Microbiol, 9(6):467-477, 2011). The modification of a single targeting RNA can be sufficient to alter the nucleotide target of a Cas protein. In some cases, crRNA and tracrRNA can be engineered as a single cr/tracrRNA hybrid to direct Cas9 cleavage activity (Jinek et al., Science, 337(6096):816-821, 2012). Like TALE nucleases, for example, the components of a CRISPR/Cas system (the Cas9 endonuclease and the crRNA and tracrRNA, or the cr/tracrRNA hybrid) can be delivered to a cell in a geminivirus-based replicon.

The coding sequence for an endonuclease can be operably linked to a promoter that is inducible, constitutive, cell specific, or activated by alternative splicing of a suicide exon. Strong, constitutive promoters may be particularly useful. Such promoters include, without limitation, the maize ubiquitin promoter (ZmUbi) used in the experiments described below, the ubiquitin promoter from Panicum virgatum (PvUbi), the ubiquitin 10 (Ubi10) promoter from Arabidopsis thaliana, the Actin 1 (Act-1) promoter from rice, the nopaline synthase (nos) promoter, octopine synthase (ocs) promoter, and mannopine synthase (mas) promoters from Agrobacterium tumefaciens, and the 35S promoter from cauliflower mosaic virus. The plant can be infected with a viral replicon containing a repair template, and the endonuclease can be expressed to cleave the DNA at the target sequence, facilitating HR on either side of the repair template to be integrated.

One or more endonuclease coding sequences can be contained in the same geminivirus construct as one or more repair templates, or can be present in one or more vectors that are separately delivered to the plant, either sequentially or simultaneously with the geminivirus construct containing the repair template(s). In some embodiments, for example, plants can be transfected or infected with a second viral vector, such as an RNA virus vector (e.g., a tobacco rattle virus (TRV) vector, a tobacco mosaic virus (TMV) vector, a potato virus X (PVX) vector, a pea early-browning virus (PEBV) vector, a wheat streak mosaic virus (WSMV) vector, or a barley stripe mosaic virus (BSMV) vector) that encodes the endonuclease. As an example, TRV is a bipartite RNA plant virus that can be used to transiently deliver protein coding sequences to plant cells. For example, the TRV genome can be modified to encode a ZFN or TALE nuclease by replacing TRV nucleotide sequence with a subgenomic promoter and the ORF for the endonuclease. The inclusion of a TRV vector can be useful because TRV infects dividing cells and therefore can modify germ line cells specifically. In such cases, expression of the endonuclease encoded by the TRV can occur in germ line cells, such that HR at the target site is heritable.

In some embodiments in which a geminivirus vector contains both a repair template and an endonuclease encoding sequence, the geminivirus can be deconstructed such that it encodes only the proteins needed for viral replication. Since a deconstructed geminivirus vector has a much larger capacity for carrying sequences that are heterologous to the virus, the repair template may be longer than 800 nt. An exemplary system using a deconstructed vector is described in the Example section below.

The construct(s) containing one or more repair templates and one or more endonuclease encoding sequences can be delivered to a plant cell using, for example, biolistic bombardment. In some cases, the one or more repair templates and endonuclease coding sequences can be delivered using Agrobacterium-mediated transformation, insect vectors, grafting, or DNA abrasion, according to standard methods.

After a plant is infected or transfected with a repair template (and, in some cases, an endonuclease encoding sequence), any suitable method can be used to determine whether GT has occurred at the target site. In some embodiments, a phenotypic change can indicate that a repair template sequence was integrated into the target site. Such is the case for the plants that were modified with geminivirus replicons containing sequences encoding fluorescent reporters, as described below, or sequences encoding herbicide or antibiotic resistance for selection and regeneration of the modified cells. In some cases, the first GT event (e.g., the insertion of a fluorescent marker or a nucleic acid conferring herbicide resistance or antibiotic resistance downstream from an endogenous promoter) can be used to select for a second or second and third gene editing (NHEJ or GT) event. PCR-based methods and sequencing methods also can be used to determine whether a genomic target site contains a repair template sequence, and/or whether precise recombination has occurred at the 5′ and 3′ ends of the repair template.

The invention will be further described in the following examples which does not limit the scope of the invention described in the claims.

### Example

### High Efficiency Gene Targeting in Hexaploid Wheat

### Materials and Methods

Vector Construction.

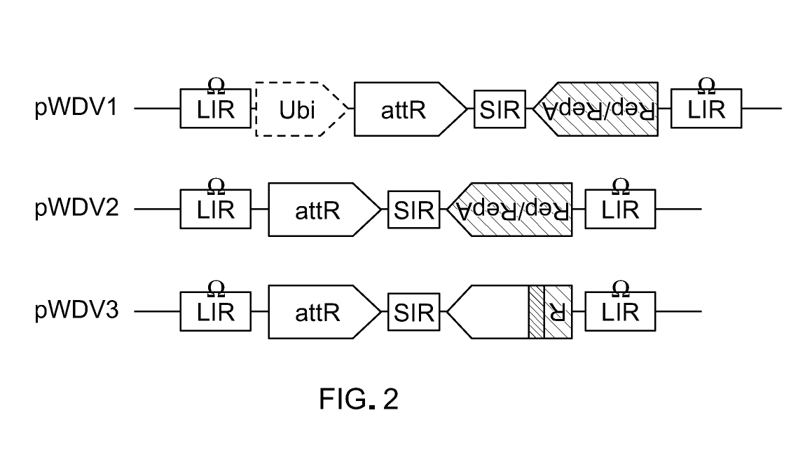

The replication elements of WDV (LIR, SIR, and Rep/RepA) were PCR amplified from pWI11 (Ugaki et al., supra) and cloned by Gibson assembly (New England Biolabs; Ipswich, Mass.) into the multi-cloning site of the pCLEAN-G185 binary vector in a LIR-SIR-Rep-LIR configuration (FIG. 2). The final vector included the Gateway attR1 and attR2 sites (Invitrogen; Waltham, Mass.) either between the ZmUbi promoter and the short intergenic region (SIR) sequence (vector pWDV1) or between the LIR and SIR sequences (vectors pWDV2 and pWDV3). The Gateway sites make it possible to clone heterologous sequences on the virion sense strand. The bi-directional LIR promoter was included in all pWDV vectors to allow release of the circular replicon. The replicase proteins (Rep/RepA) were expressed in all cases by the LIR promoter. The LIR was used to express heterologous sequences in pWDV2 and pWDV3.

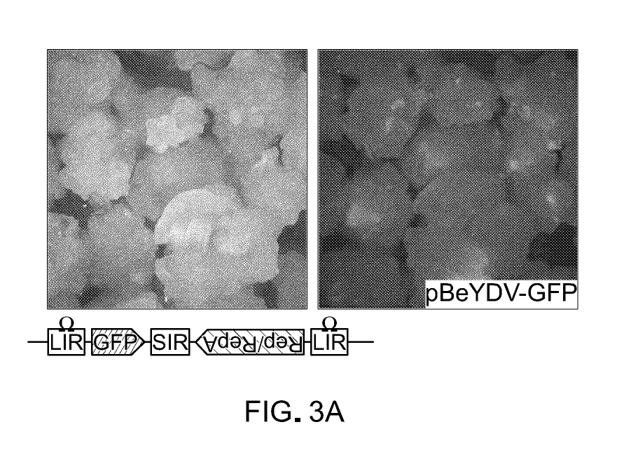

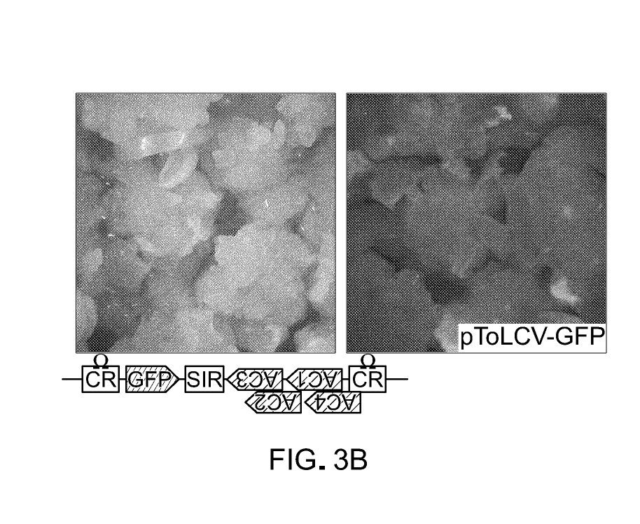

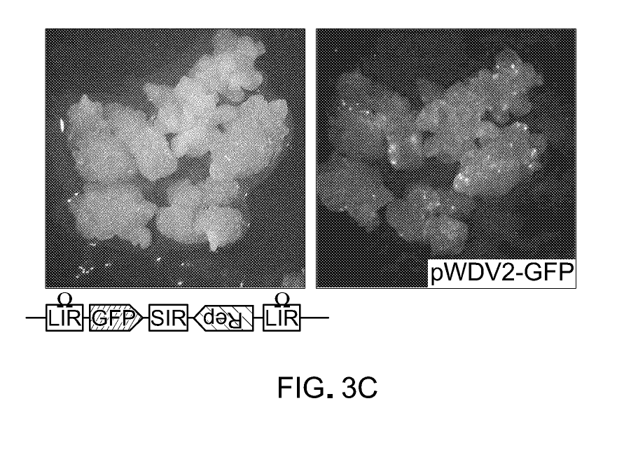

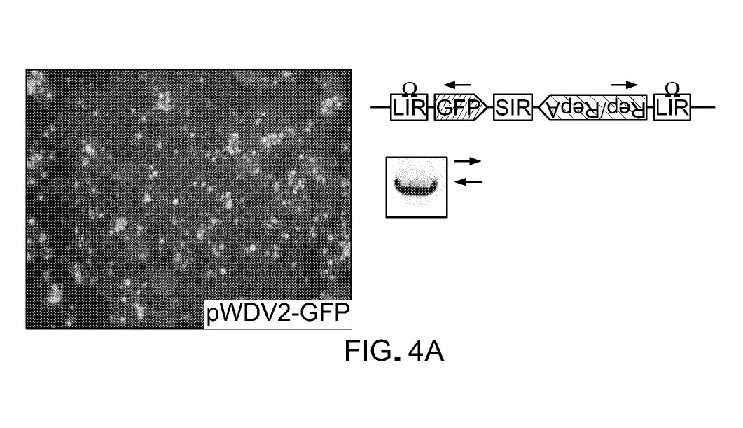

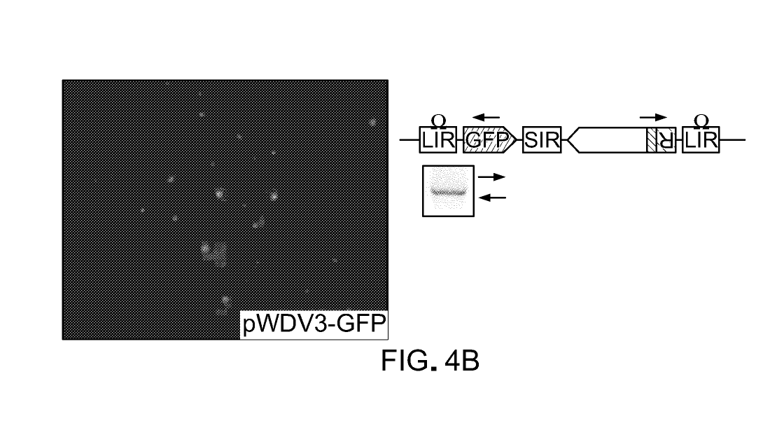

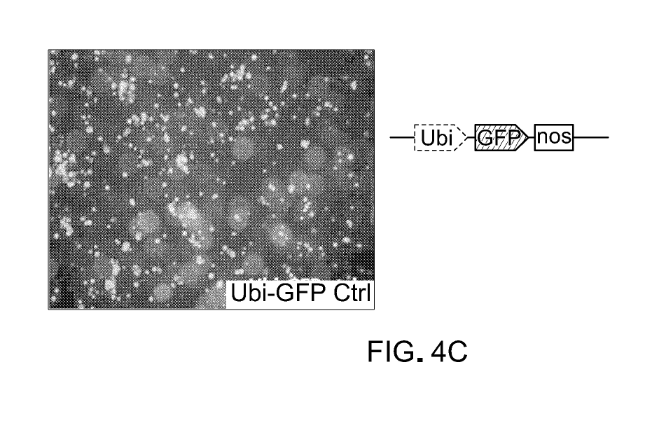

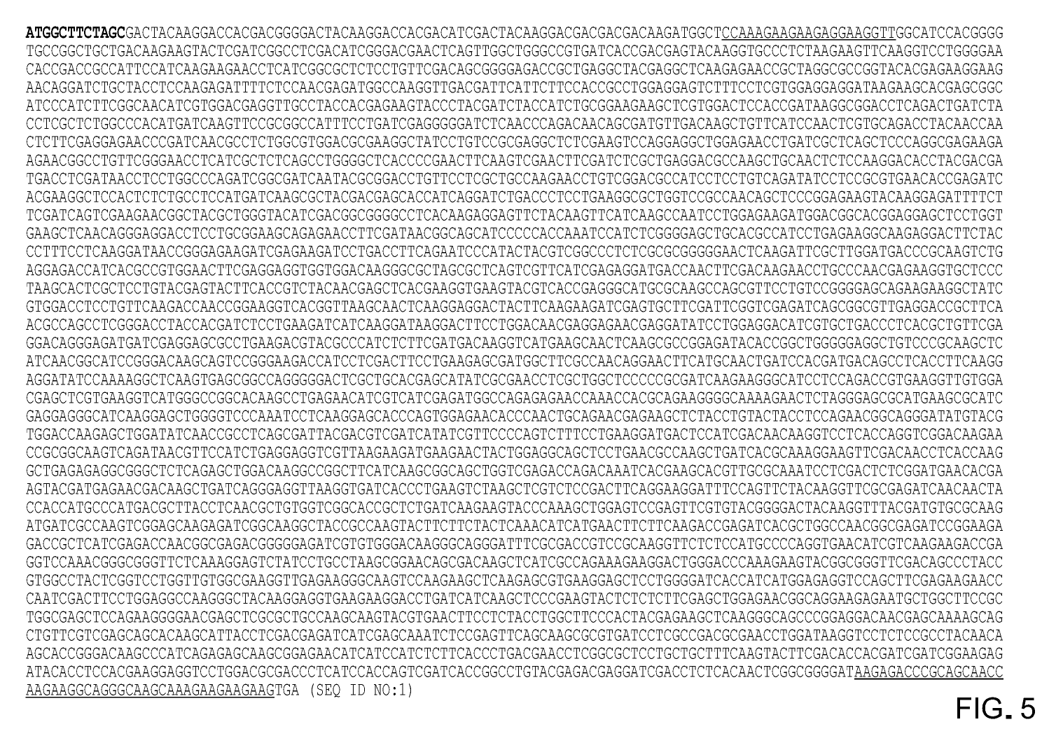

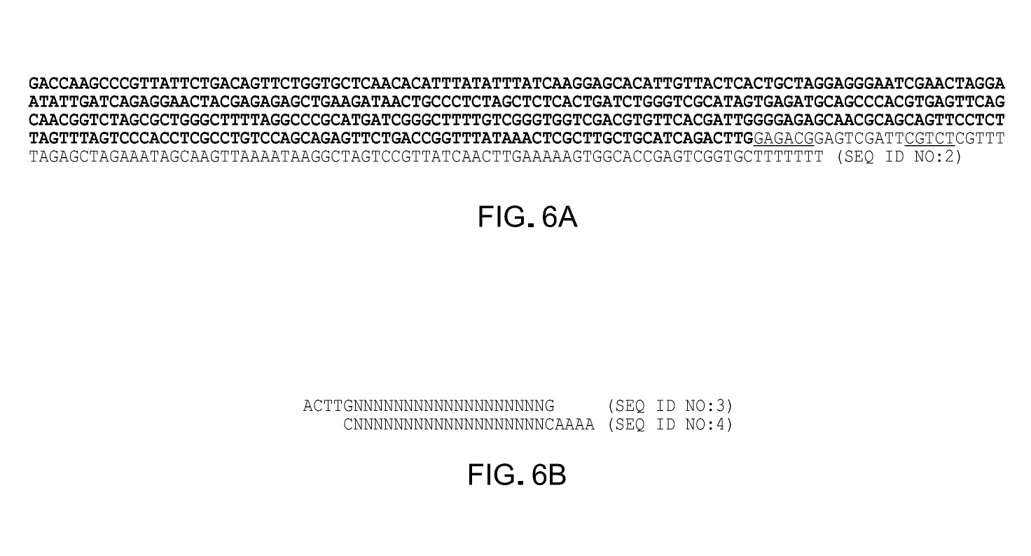

GFP was cloned into the Gateway site of the different WDV replicons, resulting in pWDV1-GFP, pWDV2-GFP, and pWDV3-GFP. For the expression experiments, the vector Ubi-GFP, having the ZmUbi promoter driving expression of GFP (FIGS. 3 and 4), was used as a non-geminivirus control. For the directed mutagenesis and GT experiments, CRISPR/Cas9 reagents were cloned into the Gateway site. A wheat-codon optimized Cas9 sequence (TaCas9) was synthesized using gBLOCKs (Integrated DNA Technologies; Coralville, Iowa) (FIG. 5). The TaCas9 coding sequence contains an N-terminal 3× Flag and N- and C-terminal nuclear localization signals (NLS) from the simian vacuolating virus 40 (SV40) and nucleoplasmin, respectively. The Triticum aestivum U6 RNA polymerase III promoter (TaU6) (bold, FIG. 6A) was used for expression of the sgRNAs. sgRNAs were designed to recognize 20 nt (G-N19) in the target sites, and a PAM (Protospacer Adjacent Motif) sequence (NGG) was adjacent to the 3′ end. sgUbi1 (GCTGGTGCAACTGGTGGCCC; SEQ ID NO:27) was completely complementary to the third exon of the ubiquitin homeoalleles on chromosomes 1AL and 1DL; it had one mismatch at position 6 (from the PAM sequence) with the 1BL target site. sgMLO1 (GAACTGGTATTCCAAGGAGG; SEQ ID NO:28) was complementary to the 5AL and 4DL homeoalleles of the MLO gene, and had 1 mismatch at position 6 from the PAM sequence to the homeoallele on chromosome 4BL. sgEPS1 (GTAATGCTGGAACTGCAATG; SEQ ID NO:29) had complete complementarity with exon 1 of the EPSPS alleles in both the 4AL and 7DS chromosomes.

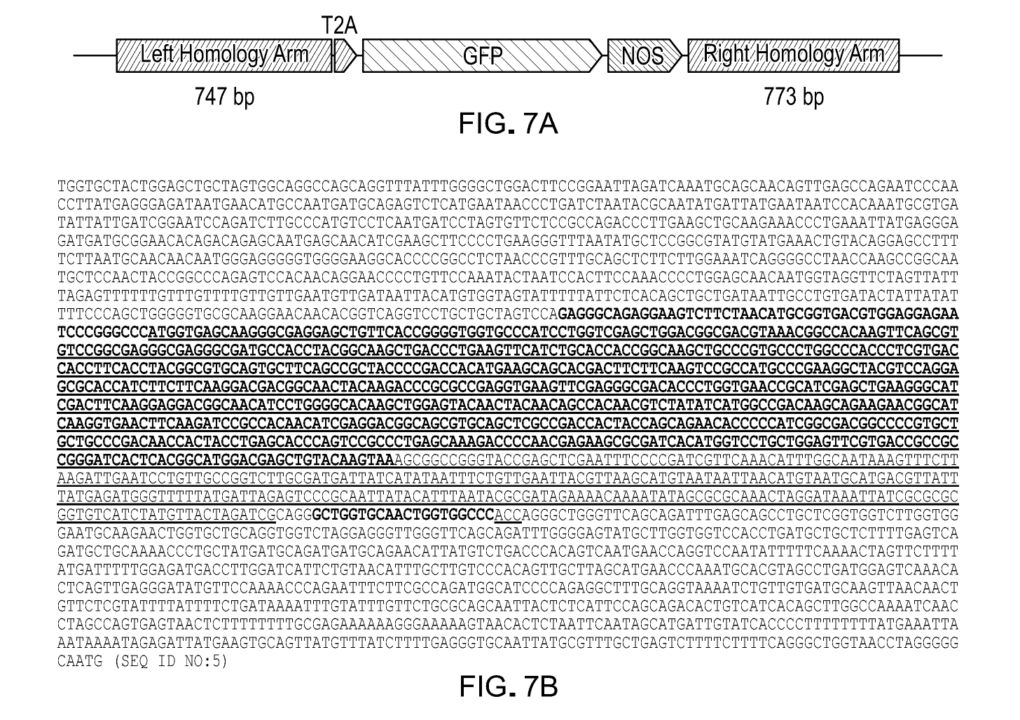

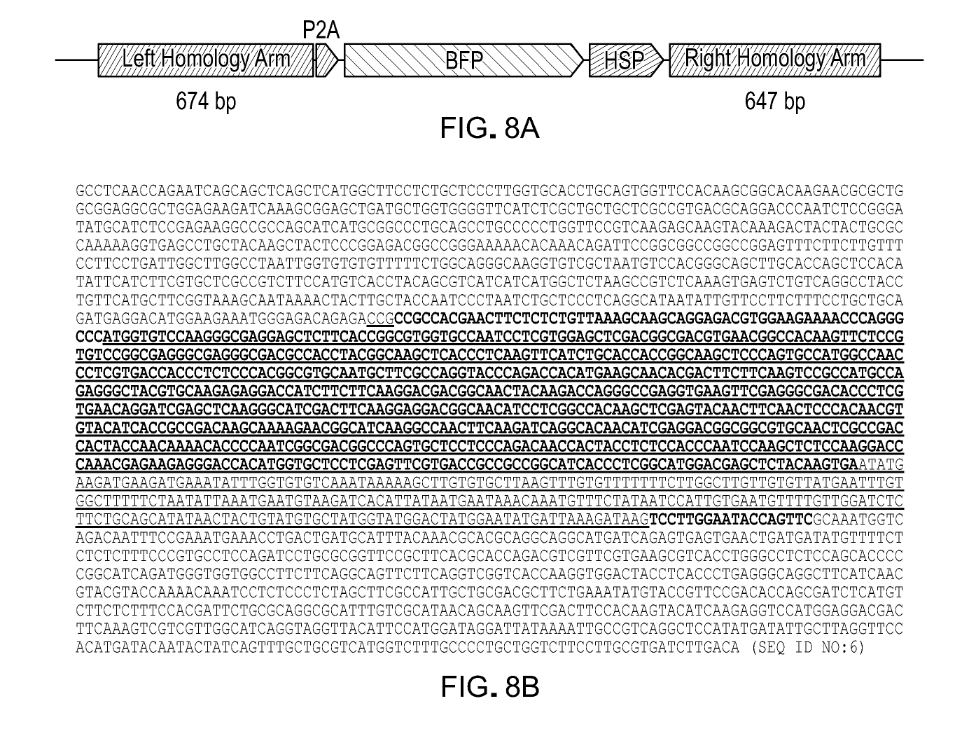

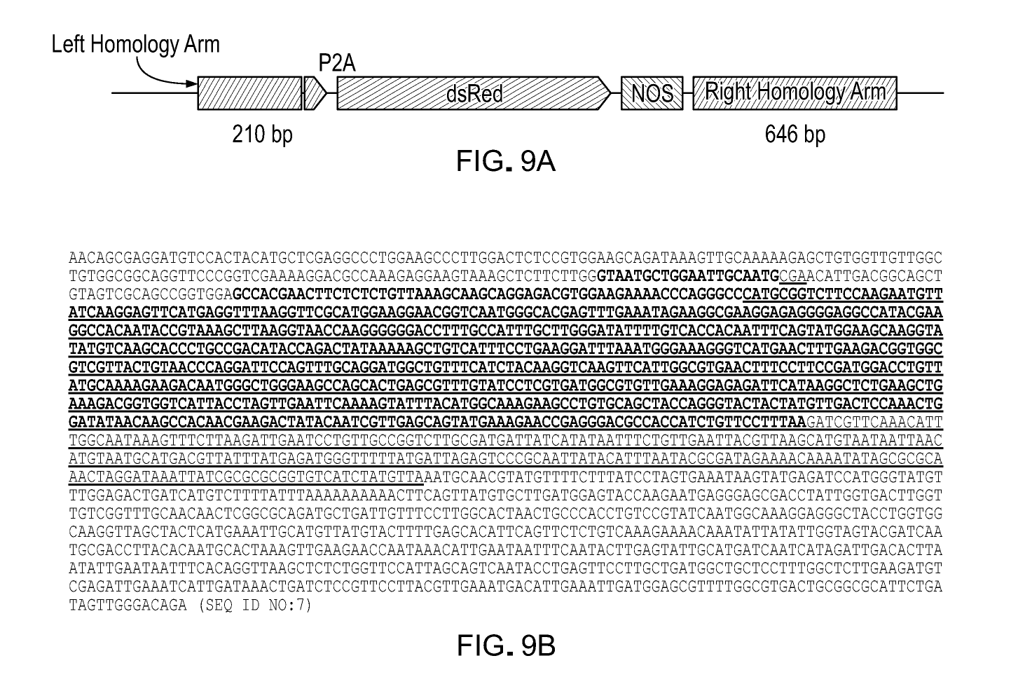

Donor templates for GT experiments were generated to knock-in the different fluorescence reporters into the ubiquitin, MLO, and EPSPS loci. GT into the ubiquitin gene was performed using a promoter-less ‘P2A-gfp-nos terminator’ cassette (referred as T2A:gfp) (FIGS. 7A and 7B). The cassette was flanked by left and right homology arms (PCR amplified from the 1DL homeoallele) of 747 and 773 bp, respectively. For GT into the MLO gene, the ‘P2A-bfp-HSP terminator’ sequence (referred to as P2A:bfp, FIGS. 8A and 8B) was synthesized, flanked by 674 bp and 647 bp homology arms. The homology arms were PCR amplified from the 4DL homeoallele. For the GT experiments into the EPSPS gene, a promoter-less ‘P2A-dsRed-nos terminator’ (referred as P2A:dsRed, FIG. 9A) flanked by 210 bp and 646 bp homology arms cloned from the 4AL homeoallele was synthesized. Silent mutations in the PAM sequence of each designed sgRNA were introduced, when necessary, into the donor templates to avoid cleavage by the CRISPR/Cas reagents.

Plant Material.

Wheat (T. aestivum cv Bobwhite) plants were grown at 20° C. (day) and 14° C. (night) temperatures with a relative air humidity of 60% under a 16 hour photoperiod. Plants of the maize (Zea mays) Hi II hybrid genotype were grown in the greenhouse at 28° C. with light supplementation for a 12 hour photoperiod. Rice (Oryza sativa cv Nipponbare) grains were dehulled and sterilized with 75% ethanol and 2.5% sodium hypochlorite and then plated on ½ MS solid medium in a round glass cup, and covered with sterilized plastic film. Plants were grown at 28° C. with a photoperiod of 16 hours light and 8 hours dark for about 14-20 days in a growth chamber. Wheat and rice protoplast isolation was carried out from wheat and rice leaves as described elsewhere (Shan et al., supra).

Biolistic Transformation.

Immature wheat scutella (0.5-1.5 mm) were isolated from primary tillers harvested 16 days after anthesis and used for biolistic transformation about 1 hour after isolation, or cultured for 2-3 weeks to induce callus. Scutella isolation and culture conditions were as described elsewhere (Gil-Humanes et al., “Genetic transformation of wheat: Advances in the transformation method and applications for obtaining lines with improved bread-making quality and low toxicity in relation to celiac disease.” In Genetic Transformation. Ed. Alvarez, InTech; 2011:135-150) with an osmotic treatment applied between 1 hour before and 2 hours after bombardment. F₂ immature zygotic embryos (1.5 to 2.0 mm) of corn were aseptically dissected from ears harvested 10 to 13 days post pollination. Corn immature embryos were isolated as described elsewhere (Ishida et al., Nat Protoc 2007, 2:1614-1621) and placed with the embryo axis facing down in culture medium to induce cell division and callus formation for 2-3 weeks. Biolistic bombardment of immature embryos or calli of the different species was carried out using a PDS-1000 gene gun. Equimolar amounts (1 pmol DNA mg-1 of gold) of each plasmid were used for each experiment with 60 μg of gold particles (0.6 μm diameter) per shot. GFP images of transformed tissue were taken using a camera mounted on a Nikon microscope.

Genomic DNA and Total RNA Isolation from Wheat Callus and Protoplasts.

Total genomic DNA was isolated from ˜200 mg of wheat callus with a 20 mM Tris-HCl (pH 7.5), 250 mM NaCl, 25 mM EDTA, 0.5% SDS extraction solution that included RNase. A 2% cetyl trimethylammonium bromide (CTAB) solution was used for total genomic DNA isolation from leaves (˜50 mg tissue) and protoplasts (200,000 cells). RNA from wheat callus (˜200 mg) was isolated using TRIzol reagent (Invitrogen) according to the manufacturer's instructions, and treated with TURBO DNase (Ambion; Waltham, Mass.) to eliminate DNA contamination. RNA (500 ng) was converted to cDNA using the High-Capacity cDNA Reverse Transcription Kit (Applied Biosystems; Foster City, Calif.).

Detecting WDV Replicon Circularization.

Circularization of the replicon was detected by PCR using the Expand Long Template system (Roche; Basel, Switzerland). Specific primers were designed to detect circularization of the different pWDV constructs expressing GFP or TaCas9 (TABLE 1). PCR conditions in all the cases were: 50-75 ng of DNA template, 0.15 μM of each primer, lx Expand Long Template Buffer 1, and 1.87 U of the enzyme mix in a 25 μl reaction. Cycling conditions consisted of an initial denaturation step of 94° C. for 5 minutes followed by 30 cycles of 94° C. for 30 seconds, 55° C. for 30 seconds, and 68° C. for 1 minute, followed by a final extension step of 68° C. for 5 minutes.

Quantitative Real Time PCR (qRT-PCR).

qRT-PCR was used to assess both DNA copy number and gene expression from the replicons. qRT-PCR was performed using the FastStart Universal SYBR Green Mix kit (Roche) on the LIGHTCYCLER® 480 Instrument (Roche). To carry out copy number and relative gene expression experiments, primers were designed for the GFP coding sequence within the WDV constructs (GFP1_F and GFP1_R), for the Ubi-GFP control plasmid (GFP2_F and GFP2_R), and for the Rep and RepA coding sequence (Rep_F and Rep_R) (TABLE 1). The actin gene (actin_F and actin_R) and the RLI (similar to A. thaliana RNase L inhibitor protein) gene (RLI_F and RLI_R) were used as references to normalize replicon copy number and gene expression. qRT-PCR conditions were: 0.3 μM of each primer and 1× FastStart Universal SYBR Green Mix in a final volume of 10 μl, with either cDNA obtained from 20 ng of total RNA or 50 ng of total genomic DNA for quantification of gene expression and copy number, respectively. Primer efficiencies and Cq values were determined using the LingRegPCR v2013.0 software (Ruijter et al., Nucleic Acids Res 2009, 37:e45). Normalized copy number and gene expression were calculated using an equation described elsewhere (Hellemans et al., Genome Biol 2007, 8) for multiple reference genes, and the results were standardized using an adapted version of the Microsoft Excel Qgene template (Muller et al., BioTechniques 2002, 32:1372-1379). Three technical replicates were performed for each sample. Error bars in the figures represent standard errors of three different biological replicates (DNA or RNA from calli or protoplasts transformed independently).

Molecular Characterization of Targeted Mutagenesis.

A PCR/restriction enzyme assay was performed to detect mutations induced by NHEJ in the ubiquitin gene in transfected protoplasts. Insertions or deletions at the DSB induced by sgUbi1 would result in loss of a HaeIII restriction site located just upstream of the PAM sequence. A 533-bp fragment containing the sgUbi1 target site was PCR amplified simultaneously from the 1AL, 1BL, and 1DL alleles using the Ubi_F and Ubi_R primer pair (TABLE 1). PCR conditions were 50 ng of DNA template, 0.5 μM of each primer, 1×Q5 Reaction Buffer (New England Biolabs; Ipswich, Mass.), and 0.5 U of the Q5 polymerase (New England Biolabs) in a 25 μl reaction. Cycling conditions consisted of an initial denaturation step of 98° C. for 30 seconds, followed by 40 cycles of 98° C. for 10 seconds, 64° C. for 20 seconds, and 72° C. for 20 seconds, with a final extension step of 72° C. for 5 minutes. The PCR product of each reaction was digested with HaeIII for 3 hours and resolved on a 2% agarose gel. The frequency of mutations was estimated by quantifying the intensity of the undigested and digested bands with the software ImageJ (Schneider et al., Nat Meth 2012, 9:671-675) as described elsewhere (Shan et al., Nat Protocols 2014, 9:2395-2410). Cleavage-resistant amplicons were gel purified, cloned into pJET1.2 (ThermoScientific; Waltham, Mass.) and sequenced.

Molecular Characterization of GT.

Gene targeting of each of the GFP, BFP, and dsRed reporters was detected by PCR. One primer in the genomic flanking region and one primer in the donor template were combined in each case to detect the 5′ and 3′ junctions of the insertion (TABLE 1). PCR conditions were: 150 ng of DNA template, 0.5 μM of each primer, 1×Q5 Reaction Buffer (New England Biolabs), and 0.5 U of the Q5 polymerase (New England Biolabs) in a 25 μl reaction. Cycling conditions consisted of an initial denaturation step of 98° C. for 30 seconds, followed by 40 cycles of 98° C. for 10 seconds, a variable annealing temperature for 20 seconds, and 72° C. for 45 seconds, and a final extension step of 72° C. for 5 minutes. Amplicons were separated on a 1% agarose gel, purified, and cloned into pJET1.2 (ThermoScientific) for sequencing. About 10 colonies of each transformation event were sequenced by Sanger sequencing and analyzed.

Quantifying Multiplexed GT in Wheat Protoplasts.

Multiplexed GT was calculated by dividing the number of protoplasts expressing GFP and BFP by the total number of cells, and normalizing to the transformation efficiency of each experiment. A Nikon A1 Spectral Confocal Microscope was used to collect random photos with the different filters. Image J software was used to count the number of cells (GFP- and/or BFP-expressing cells and total number of cells) in 10 random images for each treatment and experiment. Transformation efficiency was estimated with a replicon-based control plasmid expressing GFP (pWDV2-GFP).

### Results

### Design of DNA Replicons for Genome Editing in Monocots

Two different geminiviruses—WDV and ToLCV—were deconstructed to create autonomous replicons that function in plant cells. Specifically, the movement protein (MP) and coat protein (CP) coding sequences were removed from WDV and ToLCV, thereby eliminating the possibility of cell-to-cell movement as well as plant-to-plant insect-mediated transmission. The lack of the CP also increases the copy number of dsDNA replicon intermediates (Padidam et al., J Virol 1999, 73:1609-1616), likely because CP is not available to sequester and package ssDNA into virions, and loss of CP/Rep interactions represses viral replication (Malik et al., Virology 2005, 337:273-283). A GFP coding sequence was inserted into both vectors such that expression would be driven from the endogenous viral promoters (giving rise to ToLCV-GFP and pWDV2-GFP, FIGS. 3B and 3C).

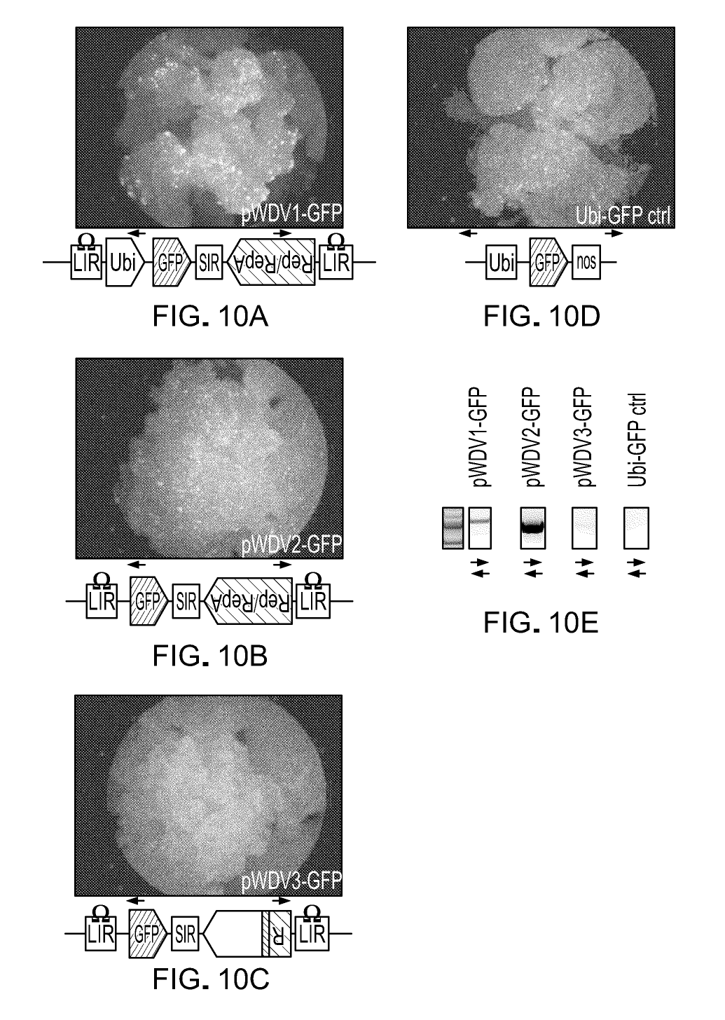

pWDV2-GFP and ToLCV-GFP were used to transform wheat calli by biolistics. As a control, calli also were transformed with a BeYDV replicon carrying a GFP cassette (pBeYDV-GFP) (Baltes et al., supra). Only cells transformed with pWDV2-GFP showed evidence of GFP expression (FIG. 3C). pWDV2-GFP was also able to replicate and express GFP in rice (FIG. 4A) and corn (FIG. 10B), as well as barley and green millet (Setaria viridis). Consequently, WDV-derived replicons were used as the platform for targeted modification of monocot genomes, focusing on wheat. Two other WDV-derived replicons were generated (FIG. 2): pWDV1 has the maize ubiquitin1 promoter (ZmUbi) downstream of the left long intergenic region (LIR) to drive expression of heterologous protein coding sequences; and pWDV3 is a replicase-deficient version of pWDV2 that has a premature stop codon in the Rep/RepA gene (C>G substitution at position 11 of the nucleotide coding sequence).

Time Course of Replicon Amplification and Gene Expression.

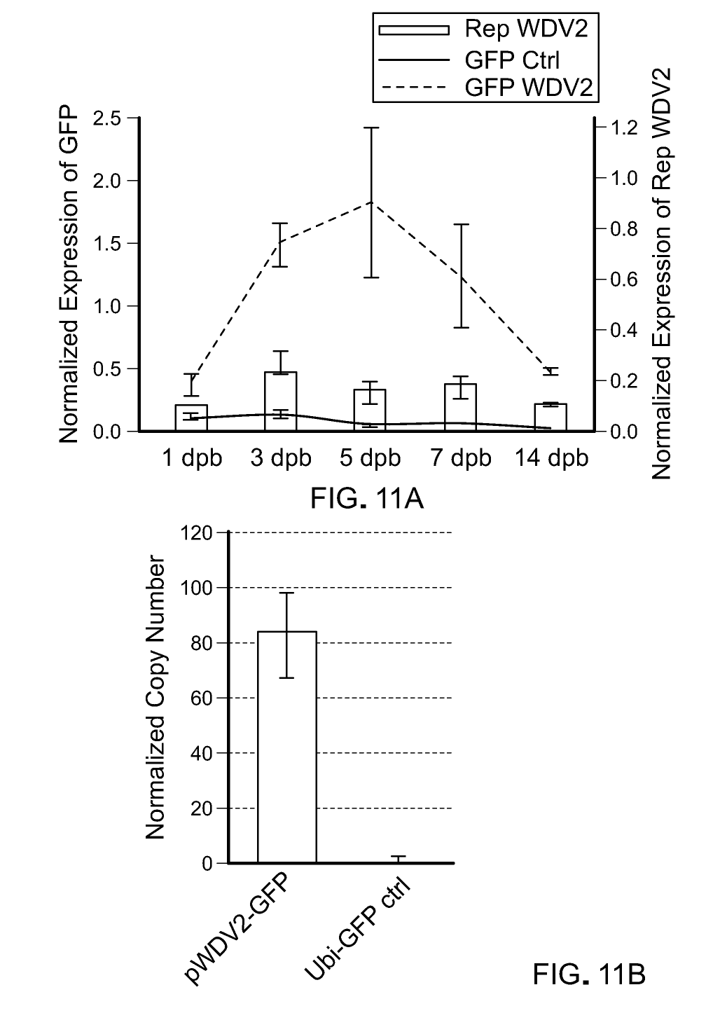

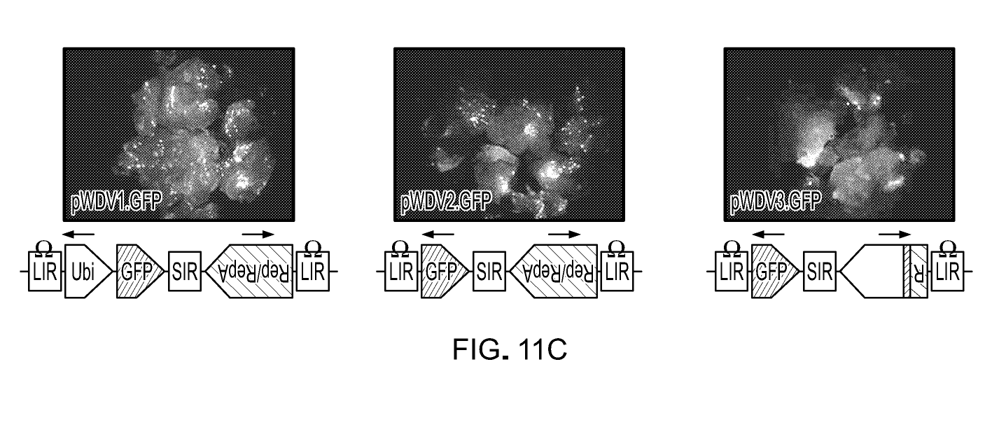

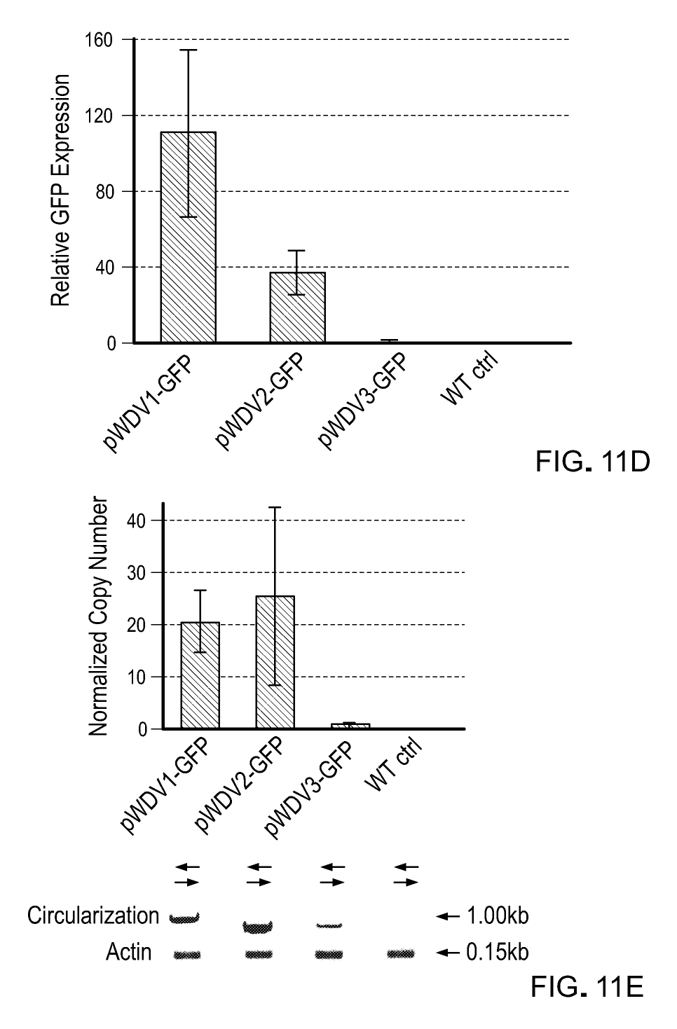

Wheat calli were transformed with either pWDV2-GFP or the Ubi-GFP control to study protein expression and replication over a 2-week time course (FIG. 11A). Relative expression of GFP and Rep/RepA transcripts was monitored by quantitative real time PCR (qRT-PCR). A peak in Rep/RepA expression was observed at about 3 days post-bombardment (dpb). GFP expressed from pWDV2-GFP peaked between 3-7 dpb and then decreased rapidly. The decrease in GFP expression may have been a consequence of viral DNA methylation Bian et al., Mol Plant Microbe Interact 2006, 19:614-624; Seemanpillai et al., Mol Plant Microbe Interact 2003, 16:429-438; and Yadav and Chattopadhyay, Mol Plant Microbe Interact 2011, 24:1189-1197), and/or post-transcriptional gene silencing mediated by the RNAi defense mechanism (Yadav and Chattopadhyay, supra; Rodriguez-Negrete et al., J Virol 2009, 83:1332-1340; and Vanitharani et al., Trends Plant Sci 2005, 10:144-151). In addition, it was found that at 5 dpb, the copy number of the pWDV2-GFP replicon was about 80 times higher than the Ubi-GFP control plasmid (FIG. 11B); GFP expression was about 30-times higher (FIG. 11A). Thus, the optimal time frame for collecting samples in genome engineering experiments was estimated to be between 5-7 days. These results were consistent with experiments using the BeYDV in infiltrated tobacco plants (Baltes et al., supra). In that case, the maximum copy number occurred around 5 days after transformation, with around 6,000 copies of the replicon per single-copy gene, which resulted in high expression of the heterologous sequence on the replicon.

Different Architectures of WDV Show Differences in Replication and the Expression of the Heterologous Proteins.

To evaluate promoter activity for heterologous protein expression, GFP expression and copy number of the replicon were monitored by qRT-PCR in wheat calli transformed with the different replicon architectures (FIGS. 11C-11E). The level of GFP expression from the LIR bi-directional promoter in pWDV2-GFP was compared with the expression from the ZmUbi promoter in the WDV1-GFP plasmid in transformed calli 5 dpb. The pWDV3-GFP vector was used in the experiment as non-replicating control. GFP expression and replicon circularization were observed in calli transformed with the three different constructs, although in the non-replicating control (pWDV3-GFP), expression was observed in only a few cells (FIG. 11C). Quantification of expression by qRT-PCR showed a 110-fold increase in GFP expression with pWDV1-GFP and a 37-fold increase with pWDV2-GFP, when normalized to the non-replicating control (pWDV3-GFP) (FIG. 11D). The copy number of each plasmid was then analyzed (FIG. 11E), revealing a very similar increase in both pWDV1-GFP (20-fold) and pWDV2-GFP (26-fold) compared to the non-replicating control (WDV3-GFP). Together, these results showed that the ZmUbi promoter (˜2 kb) positively influenced expression of the heterologous protein in the replicon without compromising replicon replication. Lower copy numbers of WDV vectors in maize cells have been reported when the size of the replicon was increased to ˜3 kb (Timmermans et al., Nucleic Acids Res 1992, 20:4047-4054). In the present system, however, the extra ˜2 kb of the ZmUbi promoter did not significantly affect replication. Consequently, the pWDV1 architecture was selected for gene targeting experiments, since both high expression of the nuclease and a high copy number of the donor template are required for GT.

WDV-Induced Targeted Mutagenesis.

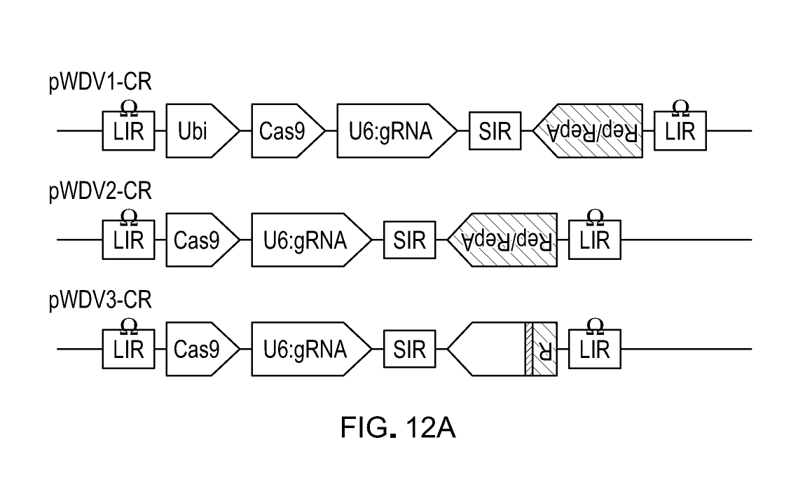

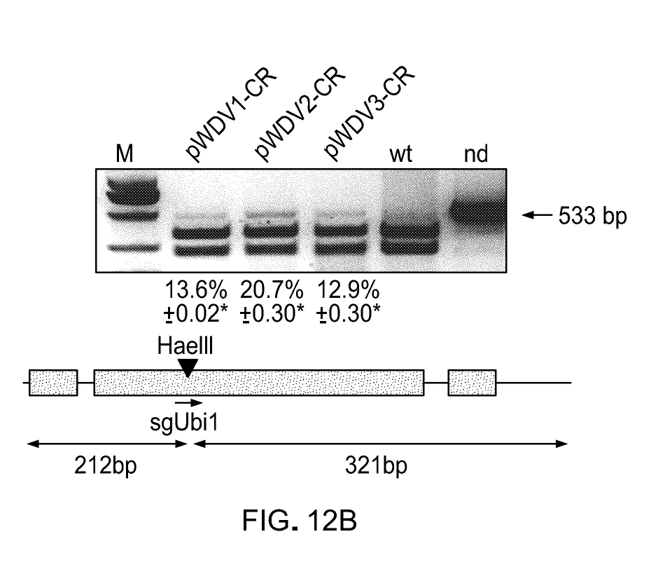

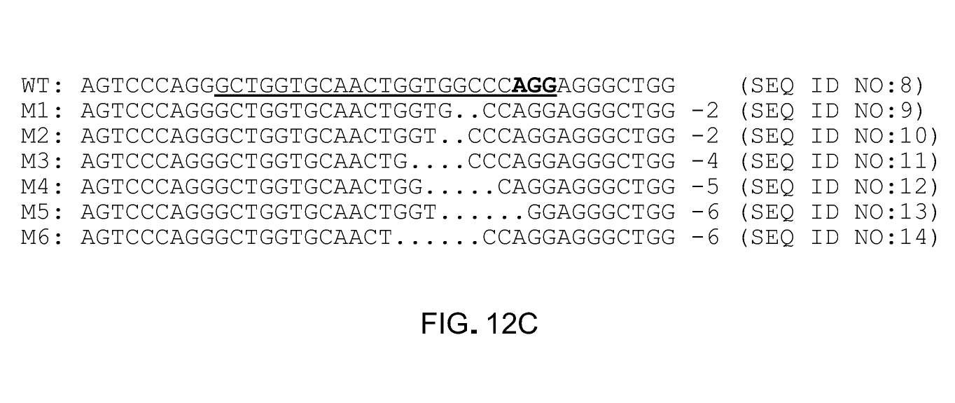

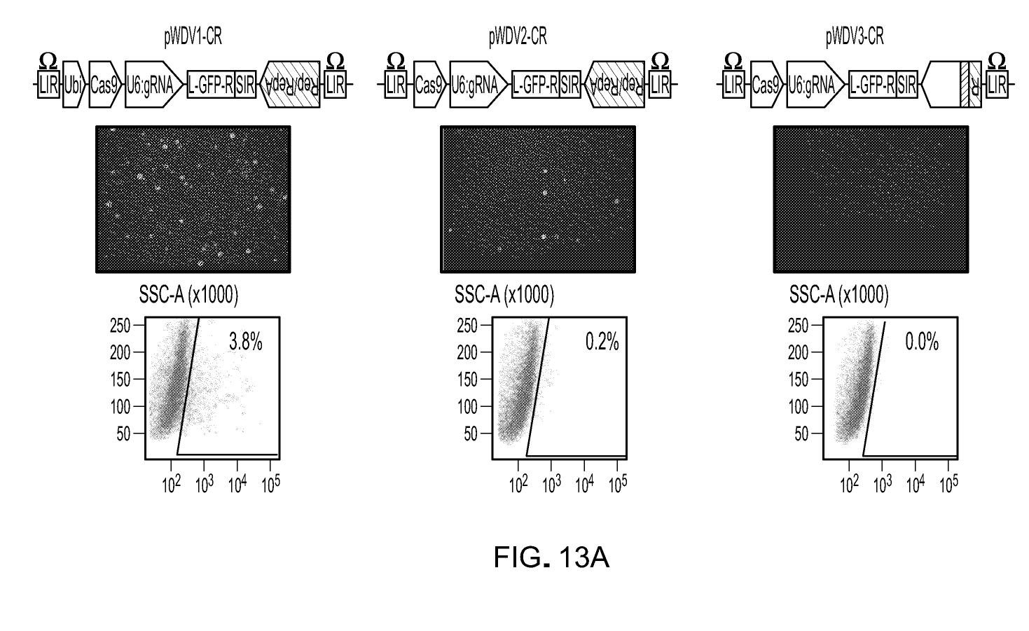

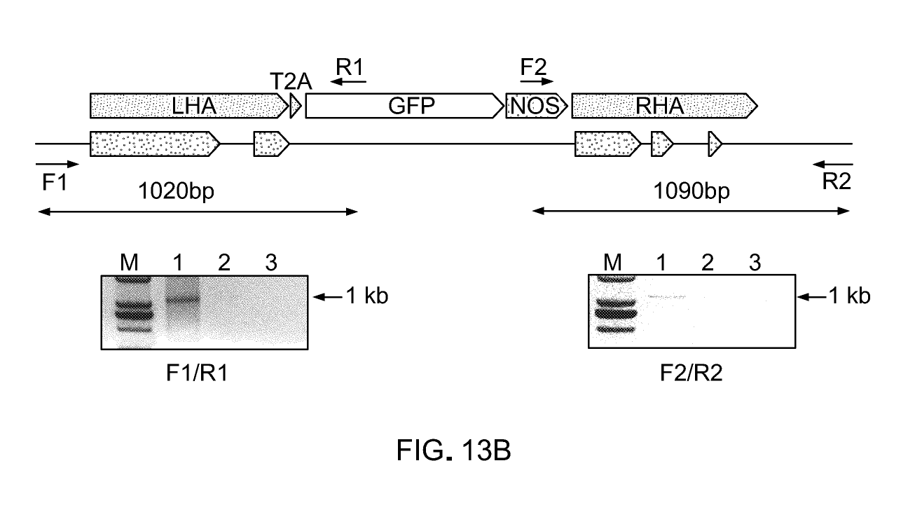

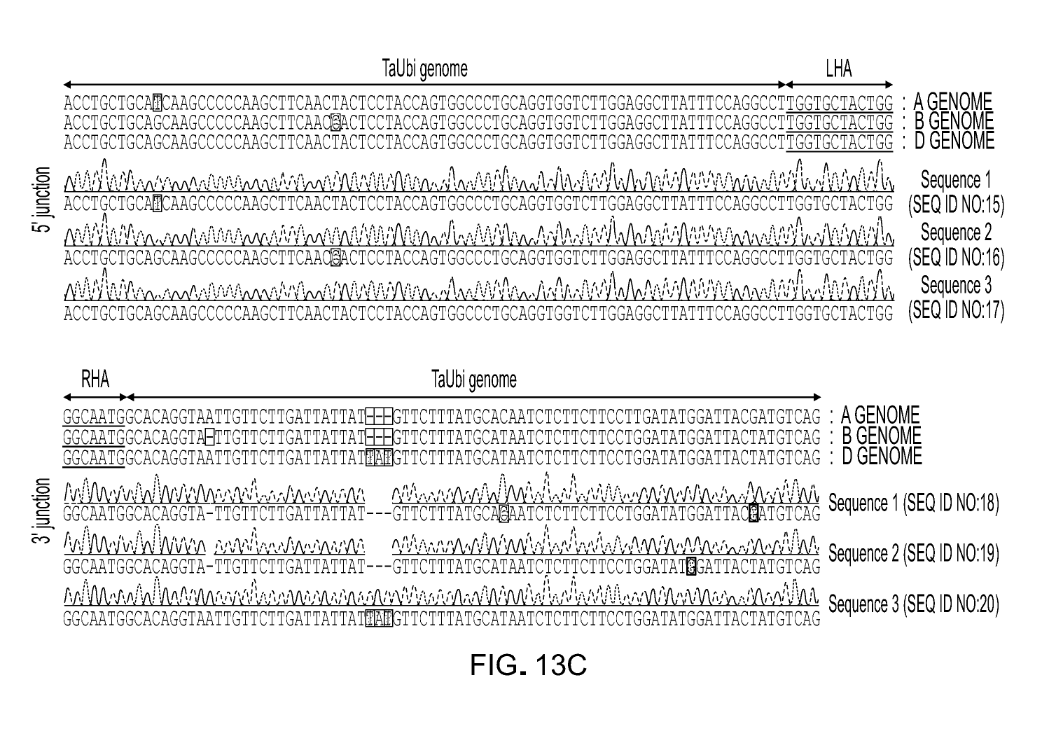

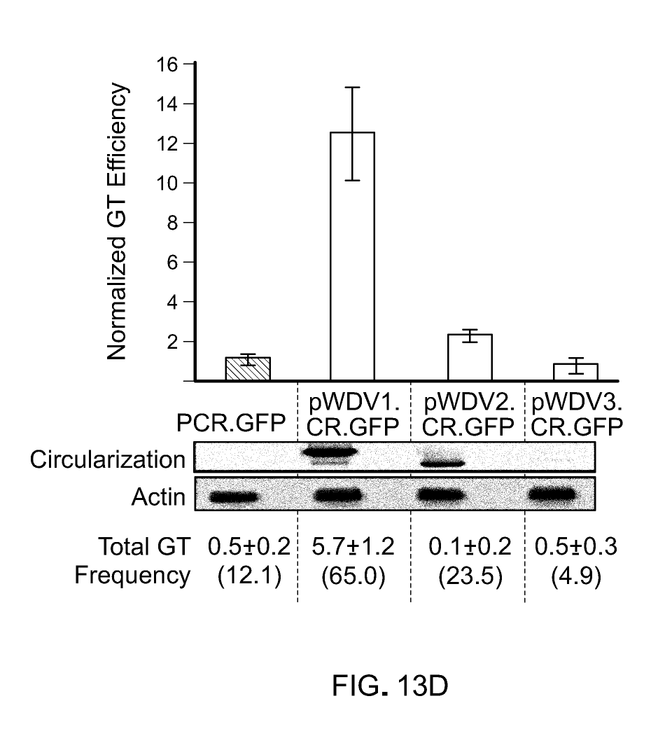

To test whether the WDV-derived replicons enable targeted mutagenesis, a 20 nt chimeric single-guide RNA (sgRNA) that recognizes the third exon of the ubiquitin gene (sgUbi1) was designed. Wheat protoplasts were transformed with different WDV constructs expressing CRISPR/Cas reagents, namely sgUbi1 and a wheat codon-optimized Cas9 (TaCas9) (vectors pWDV1-CR, pWDV2-CR, and pWDV3-CR) (FIG. 12A). Total DNA was isolated two days after transfection, and a 533-bp region encompassing the cleavage site was PCR amplified and digested with HaeIII (present in the seed sequence of the sgUbi1). Mutation frequencies ranged from 12.9-20.7% (FIG. 12B). Undigested bands were then purified and sequenced, and found to contain mostly small (2-6 bp) deletions at the predicted cleavage site (FIG. 12C). Interestingly, pWDV2-CR appeared to be most effective for targeted mutagenesis of the ubiquitin locus. These results demonstrated that the WDV replicon-system is compatible with the CRISPR/Cas9 system and can be used to enable targeted mutagenesis in wheat. High efficiency gene targeting in wheat cells mediated by WDV replicons. Having identified a functional nuclease targeting the ubiquitin locus, experiments were conducted to test whether the WDV replicons can induce GT in wheat cells. First, wheat protoplasts were used to compare the different WDV architectures for their ability to knock-in a promoter-less T2A:gfp sequence (FIGS. 7A, 7B, and 13B) into the third exon of the ubiquitin gene. CRISPR/Cas9 reagents (TaCas9 and sgUbi1) and the T2A:gfp donor template were delivered by either 1) pWDV1.CR.GFP -pWDV1 with the ZmUbi promoter expressing TaCas9; 2) pWDV2.CR.GFP—the pWDV2 version with the LIR expressing TaCas9; or 3) pWDV3.CR.GFP—the replicase-deficient pWDV3 version with the LIR expressing TaCas9. GFP expression was observed only in protoplasts transfected with the pWDV1.CR.GFP and pWDV2.CR.GFP; the former showed the highest GT efficiency (3.8%) (FIG. 13A). Targeted integration of the T2A:gfp fragment was detected only in samples transfected with the pWDV1.CR.GFP (FIG. 13B). Both 5′ and 3′ junctions of the targeted T2A:gfp fragment were sequenced to confirm that GT had occurred at the expected site (FIG. 13C). Integration of the T2A:gfp sequence was observed in the three homeoalleles (A, B, and D) of the ubiquitin gene, indicating that the sgUbi1 was active in the three sites, and that the identity between the homology arms of the donor template (cloned from the D genome) and the A and B homeoalleles (94.9% and 95.1%, respectively; TABLE 2) was sufficient to promote GT. No mutations were observed in the sequenced products, suggesting the repair was perfect. The difference in GT efficiencies between pWDV1 and pWDV2 was most likely due to a higher expression level of TaCas9, since both replicons have similar copy numbers. These results confirmed that the WDV system can be used to specifically target a heterologous sequence by HR in the wheat genome.

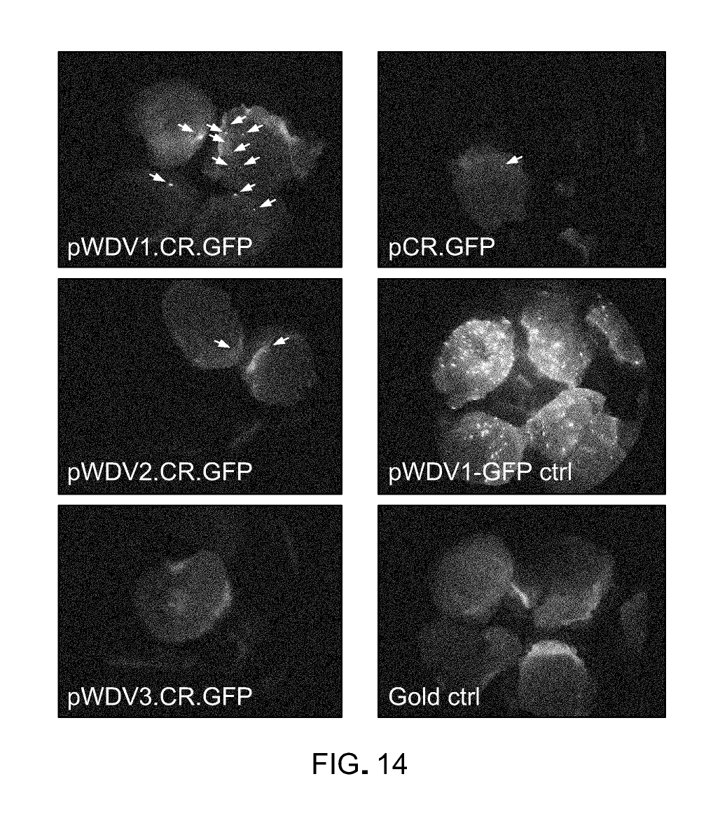

The efficiency of GT in wheat scutella with the different replicon architectures was quantified and compared with a non-viral control (pCR.GFP), in which TaCas9 is driven by the ZmUbi promoter. Wheat scutella were transformed by particle bombardment, an approach frequently used to generate transgenic wheat plants. GT events were calculated by counting the number of cells expressing GFP 7 dpb (FIGS. 13D and 14). A significant increase (˜12-fold) in the number of GFP positive cells was observed with pWDV1.CR.GFP as compared to the pCR.GFP non-replicating control and the two other architectures (pWDV2.CR.GFP and pWDV3.CR.GFP). Of the total number of scutella transformed with pWDV1.CR.GFP, 65% showed GT events, whereas only 12.1% showed GT when using the pCR.GFP control. In addition, when the total number of GT events was normalized with transformation efficiency (total number of GFP-expressing cells in the pWDV1-GFP control), the overall GT frequency of pWDV1.CR.GFP was 5.74%, and the GT frequency for the pCR.GFP control was 0.50% (FIG. 13D).

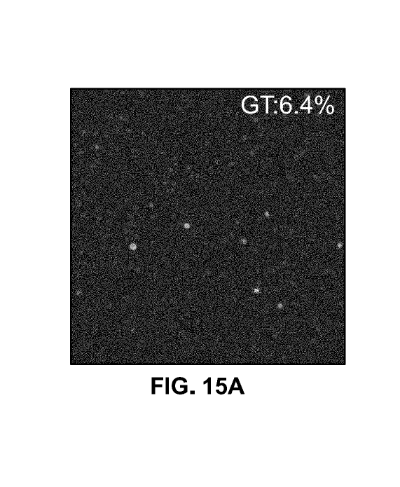

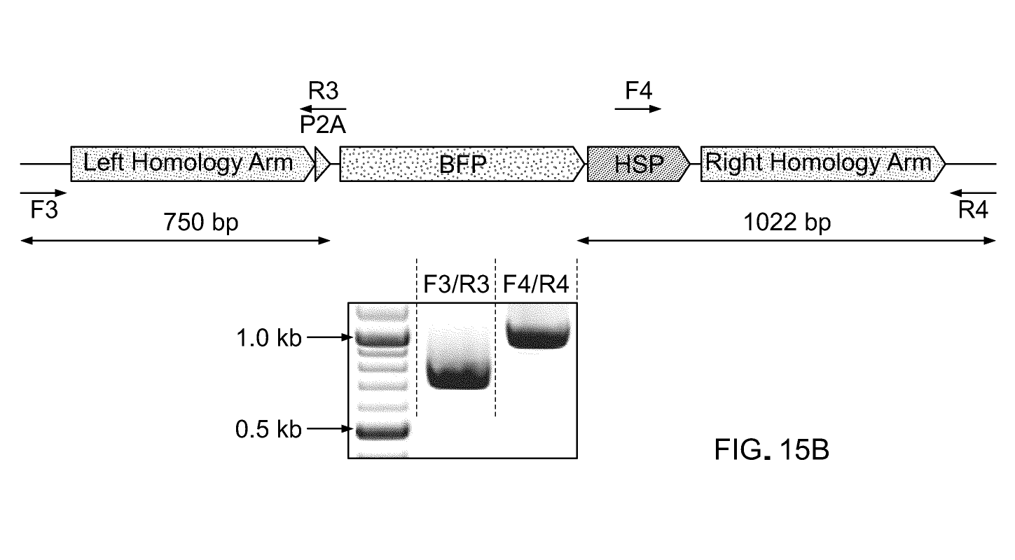

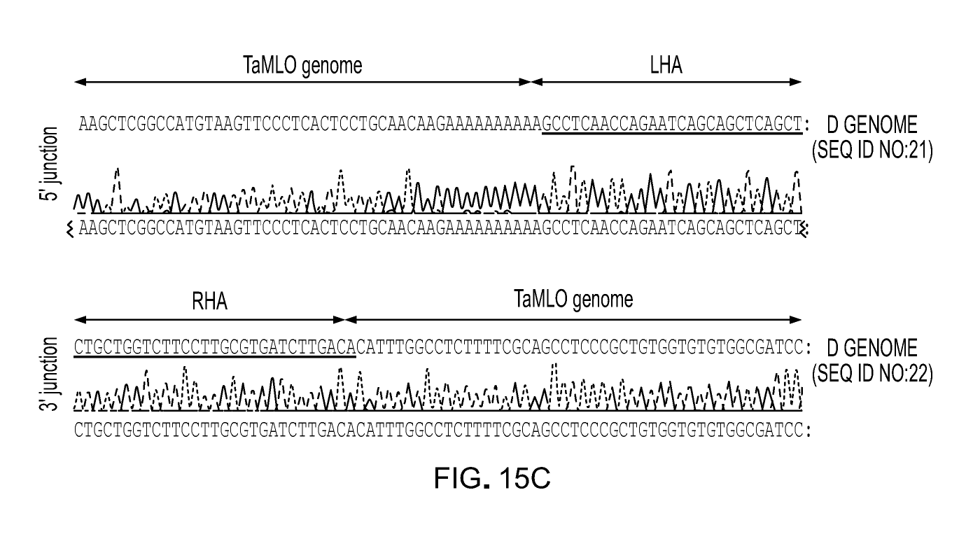

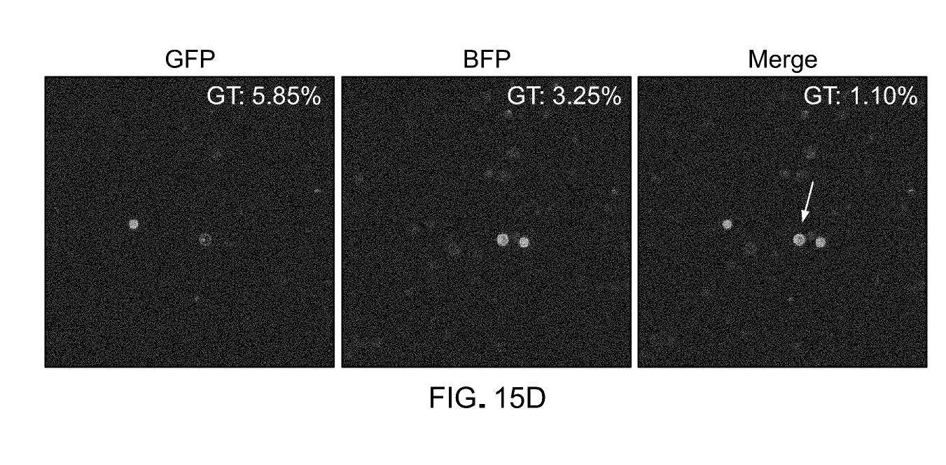

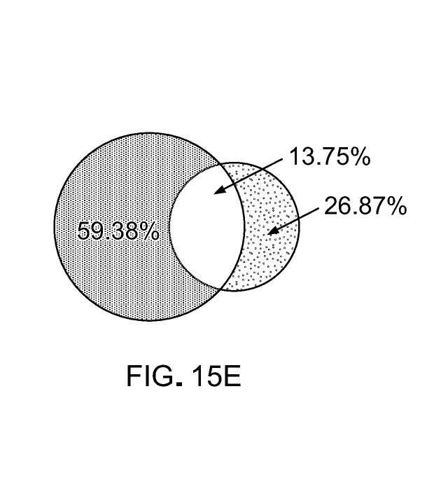

In addition, a vector containing a sgRNA complementary to the MildewLocusO (MLO) gene (sgMLO1) was synthesized and designated pWDV1.CR.BFP. A donor template designed to knock-in a promoter-less bfp coding sequence by HR also was synthesized and designated P2A-bfp (FIGS. 8A and 8B). Wheat protoplasts were transfected with pWDV1.CR.BFP, resulting in a 6.4% GT frequency (FIG. 15A). Left (5′) and right (3′) junctions were amplified by PCR (FIG. 15B), cloned, and sequenced (FIG. 15C). Integration of the P2A-bfp into the MLO homeoallele was only detected on chromosome D. The identity between the homology arms of P2A-bfp and the A and B homeoalleles (83% and 80.4%, respectively) might explain why GT with P2A:bfp occurred preferentially at the D homeoallele (100% identity). GT also may take place at the A and B homeoalleles, but at a lower frequency. These results demonstrated that the WDV system significantly enhances GT frequencies in wheat scutella, particularly when Cas9 was expressed by the strong and constitutive ZmUbi promoter.

Multiplexed Gene Targeting in Wheat Cells.

Next, the ability of the WDV replicons to achieve multiplexed GT within the same cell was examined by simultaneously targeting integration of T2A-gfp and P2A-bfp into the ubiquitin and MLO loci, respectively. Wheat protoplasts were transfected with both pWDV1.CR.GFP and pWDV1.CR.BFP, and GT frequencies of 5.85% and 3.25% were observed for the GFP and BFP reporters, respectively. The GT frequency was 1.1% for simultaneous integration of both reporters (FIG. 15C). Thus, 13.75% of the cells that underwent gene targeting contained both events (FIG. 15D).

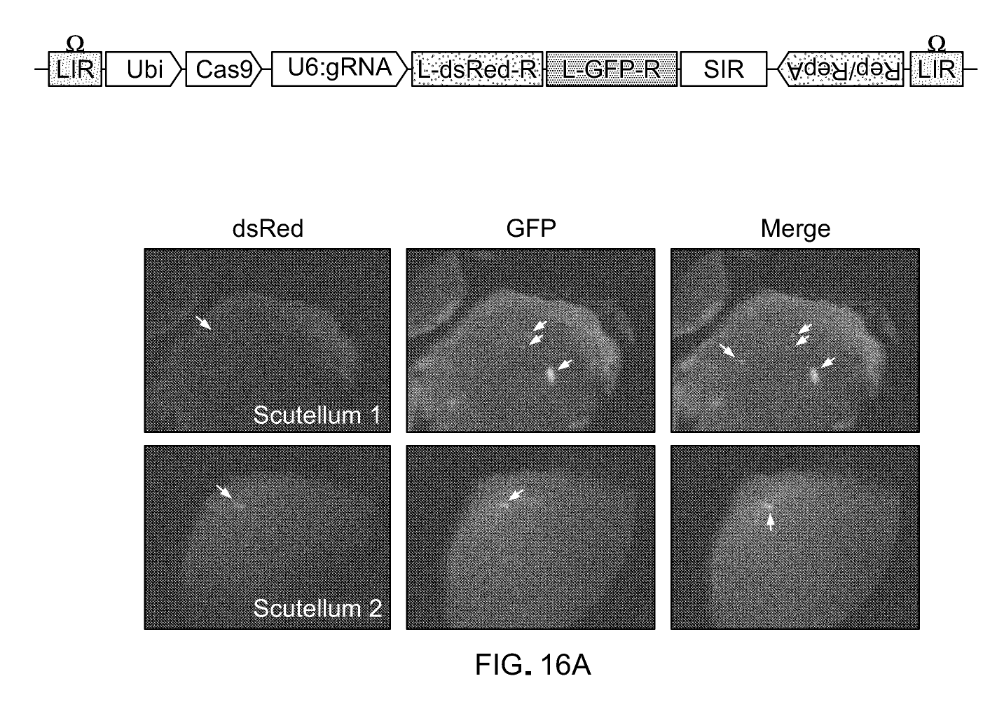

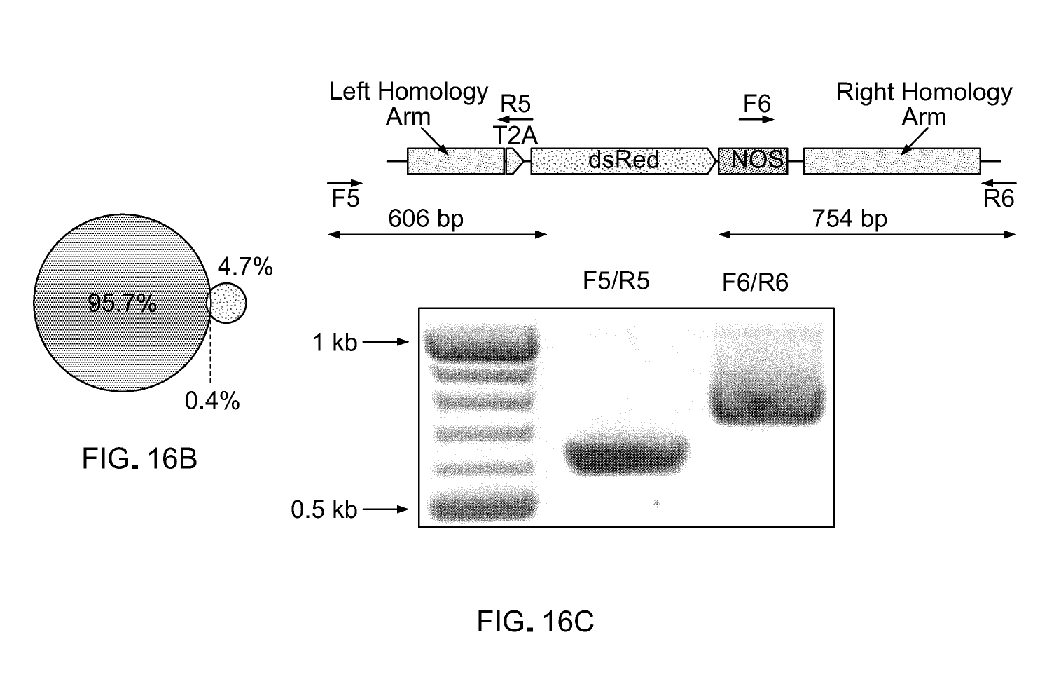

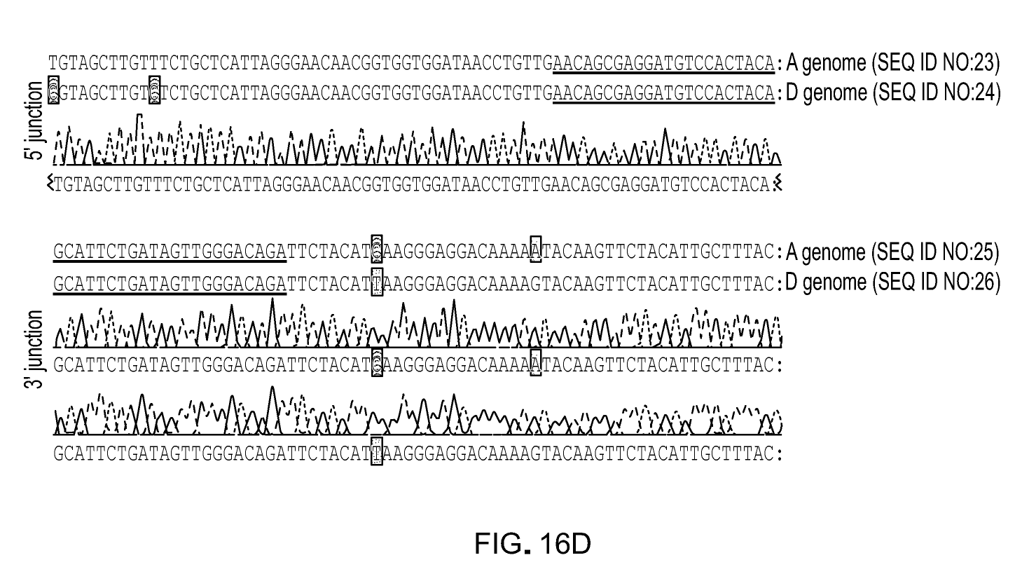

Multiplexed GT also was accomplished with a single vector (pWDV1.CR.GFP+dsRed) designed to simultaneously modify the ubiquitin and the EPSPS (5-enolypyruvylshikimate-3-phosphate synthase) loci. pWDV1.CR.GFP+dsRed has the sgUbi1 sgRNA and T2A:gfp donor template described above for GT into the ubiquitin gene, but it also carries the sgEPS1 sgRNA and a donor template designed for in-frame integration of dsRed into the EPSPS coding sequence (P2A:dsRed) (FIGS. 9A and 9B). Wheat scutella were transformed by biolistics, and multiplexed integration of both reporters was observed in 0.4% of the cells showing GT (FIGS. 16A and 16B). The low frequency of GT with the P2A:dsRed (only 4.7% of the GT cells) might be explained by the low activity of the sgRNA used (as observed in a PCR/restriction enzyme assay), the short length of the left homology arm (210-bp), or a combination of both factors. Integration of the P2A-dsRed sequence was detected in two of the EPSPS homeoalleles (A and D genomes) (FIG. 16C). Collectively, these results indicated that multiplexed GT in wheat cells can be achieved at high frequency using WDV replicons that deliver active sgRNAs and donor templates with homology arms of proper length. This may permit knock-in of a selectable marker to allow identification of cells that have undergone GT. Resistant cells could then be screened for GT at a second locus of interest, which may not provide a selectable or screenable phenotype.

Taken together, these data demonstrate that WDV-derived replicons can increase GT frequencies 12-fold over standard methods of DNA delivery, and indicate that the promoter driving Cas9 expression may be critical for achieving high efficiency gene targeting. In addition, multiplexed, targeted integration by HR was achieved in all three homeoalleles (A, B, and D) of hexaploid wheat cells using CRISPR/Cas9. The reagents described herein therefore offer considerable potential for genome editing of staple cereal crop genomes, including complex polyploid genomes such as wheat.

TABLE 1
List of primers

| | | |
|---|---|---|
| Name | Sequence (5′ to 3′) (SEQ ID NO:) | Description |

Detection of WDV replicon circularization

| | | |
|---|---|---|
| circ1_F | GCCACGAATGTTCCCCACTC (30) | Forward primer for detection of replicon circularization |
| | | with all the pWDV1 constructs and the pWDV2-GFP and pWDV3-GFP |
| circ1_R | AAAAAGGAGAACACATGCACA (31) | Reverse primer for detection of replicon circularization |
| | | with the all pWDV1 constructs |
| circ2_R | GGGACAACTCCAGTGAAAAGTT (32) | Reverse primer for detection of replicon circularization with |
| | | the pWDV2 and pWDV3 expressing GFP |
| circ3_F | ACGGAACCTGGGTGCAGATG (33) | Forward primer for detection of replicon circularization with |
| | | the pWDV2 and pWDV3 expressing CRISPR/Cas9 |
| circ3_R | CCCCGTCGTGGTCCTTGTAG (34) | Reverse primer for detection of replicon circularization with |
| | | the pWDV2 and pWDV3 expressing CRISPR/Cas9 |

qRT-PCR primers

| | | |
|---|---|---|
| Rep_F | GCTGCCAAAGACTGCAACCA (35) | Forward primer for qRT-PCR of the Rep and RepA genes in the |
| | | WDV constructs |
| Rep_R | GCCACGAATGTTCCCCACTC (36) | Reverse primer for qRT-PCR of the Rep and RepA genes in the |
| | | WDV constructs |
| GFP1_F | ATCCTCGGCCACAAGTTGGA (37) | Forward primer for qRT-PCR of the gfp gene in the WDV |
| | | constructs |
| GFP1_R | GTGGCGGGTCTTGAAGTTGG (38) | Reverse primer for qRT-PCR of the gfp gene in the WDV |
| | | constructs |
| GFP2_F | TGCAGTGCTTCAGCCGCTAC (39) | Forward primer for qRT-PCR of the gfp gene in the Ubi-GFP |
| | | construct |
| GFP2_R | TCGCCCTCGAACTTCACCTC (40) | Reverse primer for qRT-PCR of the gfp gene in the Ubi-GFP |
| | | construct |
| actin_F | GCTGGAAACGGCTAGGAGCA (41) | Forward primer for qRT-PCR of the actin housekeeping gene |
| actin_R | TGGCTGGAACAGCACCTCAG (42) | Reverse primer for qRT-PCR of the actin housekeeping gene |
| RLI_F | TTGAGCAACTCATGGACCAG (43) | Forward primer for qRT-PCR of the RLI housekeeping gene |
| RLI_R | GCTTTCCAAGGCACAAACAT (44) | Reverse primer for qRT-PCR of the RLI housekeeping gene |

Detection of IVHEI

| | | |
|---|---|---|
| Ubi_F | CCAAACCCCTGGAGCAA (45) | Forward primer for amplification of the 533 bp fragment of |
| | | the Ubiquitin gene in chromosomes |
| Ubi_R | GGTTCATGCTAAGCAACTGTG (46) | Reverse primer for amplification of the 533 bp fragment of |
| | | the Ubiquitin gene in chromosomes |

Detection of GT in the Ubiquitin locus

| | | |
|---|---|---|
| F1 | GCTTCAACTACTCCTACCAGTGGCCCTG (47) | Forward primer for detection of 5′ junction of the T2A:gfp |
| | | fragment |
| R1 | GCTTGCCGGTGGTGCAGATGAACTTCAG (48) | Reverse primer for detection of 5′ junction of the T2A:gfp |
| | | fragment |
| F2 | CCTGTTGCCGGTCTTGCGATGATTATCATA | Forward primer for detection of 3′ junction of the T2A:gfp |
| | (49) | fragment |
| R2 | GAGAACTCTGGAGCATTCACATCAAACCTG | Reverse primer for detection of 3′ junction of the T2A:gfp |
| | (50) | fragment |

Detection of GT in the MLO locus

| | | |
|---|---|---|
| F3 | ACAAGCTCGGCCATGTAAGT (51) | Forward primer for detection of 5′ junction of the P2A:bfp |
| | | fragment |
| R3 | TGCTTTAACAGAGAGAAGTTCGTG (52) | Reverse primer for detection of 5′ junction of the P2A:bfp |
| | | fragment |
| F4 | AAGTGAATATGAAGATGAAGATGAAA (53) | Forward primer for detection of 3′ junction of the P2A:bfp |
| | | fragment |
| R4 | TGAACGGTGGTTACGAGACA (54) | Reverse primer for detection of 3′ junction of the P2A:bfp |
| | | fragment |

Detection of GT in the EPSPS locus

| | | |
|---|---|---|
| F5 | TTTCTTTTCTGATGGACCCTTT (55) | Forward primer for detection of 5′ junction of the P2A:dsRed |
| | | fragment |
| R5 | TGCTTTAACAGAGAGAAGTTCGTG (56) | Reverse primer for detection of 5′ junction of the P2A:dsRed |
| | | fragment |
| F6 | AGCGCGCAAACTAGGATAAA (57) | Forward primer for detection of 3′ junction of the P2A:dsRed |
| | | fragment |
| R6 | TATCAGAAGAAGTAAAGCAATGTAGAA (58) | Reverse primer for detection of 3′ junction of the P2A:dsRed |
| | | fragment |

TABLE 2
Nucleotide sequence identity of the homology arms in the DNA donor
templates relative to the A, B, and D homeoalleles of the Ubiquitin
and MLO genes. Identity percentages were calculated using the Clustal
W Alignment tool of Geneious v7.1.9 software (Biomatters; Auckland,
New Zealand). In each case, the D genome homeoallele was used for
cloning the donor template used in the GT experiments.

| | Genomic homeoalleles | | |
|---|---|---|---|
| Homology arms | A genome | B genome | D genome |
| Ubiquitin (T2A-gfp) | 94.9% | 95.1% | 100% |
| MLO (P2A-bfp) | 83.0% | 80.4% | 100% |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for modifying genomic material of a plant cell at two or more loci, comprising:
(a) providing a plant cell that comprises two or more endogenous nucleic acid sequences to be modified;
(b) introducing into the plant cell:
(i) a first repair template targeted to a first genomic sequence within the plant cell; and
(ii) a second repair template targeted to a second genomic sequence within the plant cell; and
(c) maintaining the plant cell under conditions in which the first and second repair templates recombine by homologous recombination with their corresponding genomic loci, thereby producing a plant cell comprising targeted genomic modifications at the first and second genomic sequences.

2. The method of claim 1, wherein the first repair template is within a first geminivirus replicon that comprises, in order from 5′ to 3′, a first geminivirus long intergenic region (LIR), the first repair template, a geminivirus short intergenic region (SIR), a virus Rep/RepA coding sequence, and a second geminivirus LIR.

3. The method of claim 2, wherein the second repair template is within a second geminivirus replicon that comprises, in order from 5′ to 3′, a third geminivirus LIR, the second repair template, a second geminivirus SIR, a virus Rep/RepA coding sequence, and a fourth geminivirus LIR.

4-7. (canceled)

8. The method of claim 1, wherein the first and second repair templates are within a geminivirus replicon that comprises, in order from 5′ to 3′, a first geminivirus LIR, the first repair template, the second repair template, a geminivirus SIR, a virus Rep/RepA coding sequence, and a second geminivirus LIR.

9-10. (canceled)

11. The method of claim 1, further comprising introducing into the plant cell a first sequence specific endonuclease targeted to the first genomic sequence and a second sequence specific endonuclease targeted to the second genomic sequence, and maintaining the plant cell under conditions in which the first and second sequence specific endonucleases are expressed to introduce double stranded DNA breaks (DSBs) at the first and second genomic sequences.

12. (canceled)

13. The method of claim 1, wherein the geminivirus is wheat dwarf virus or bean yellow dwarf virus.

14. The method of claim 1, wherein the plant cell is from a polyploid plant.

15. The method of claim 14, wherein the polyploid plant is wheat, oat, triticale, tritordeum, peanut, sugar cane, white potato, tobacco, apple, banana, watermelon, canola, leek, strawberry, or cotton.

16. The method of claim 1, wherein the plant cell is a protoplast.

17. The method of claim 1, wherein all alleles and homeoalleles of the two or more endogenous nucleic acid sequences are modified in the plant cell comprising the targeted genomic modifications.

18-19. (canceled)

20. The method of claim 1, further comprising introducing into the plant cell a third repair template targeted to a third genomic sequence within the plant cell, and maintaining the plant cell under conditions in which the third template recombines by homologous recombination with its corresponding genomic sequence.

21. The method of claim 20, wherein the third repair template is within a third geminivirus replicon that comprises, in order from 5′ to 3′, a fifth geminivirus LIR, the third repair template, a third geminivirus SIR, a virus Rep/RepA coding sequence, and a sixth geminivirus LIR.

22. The method of claim 20, wherein the first, second, and third repair templates are within a geminivirus replicon that comprises, in order from 5′ to 3′, a first geminivirus LIR, the first repair template, the second repair template, the third repair template, a first geminivirus SIR, a virus Rep/RepA coding sequence, and a second geminivirus LIR.

23. The method of claim 20, further comprising introducing into the plant cell a first sequence specific endonuclease targeted to the first genomic sequence, a second sequence specific endonuclease targeted to the second genomic sequence, and a third sequence specific endonuclease targeted to the third genomic sequence, and maintaining the plant cell under conditions under which the first, second, and third sequence specific endonucleases are expressed to introduce DSBs at the first, second, and third genomic sequences.

24-26. (canceled)

27. A nucleic acid comprising a first sequence that comprises, in order from 5′ to 3′, a first geminivirus LIR, a first repair template targeted to a first genomic sequence within a first of two or more endogenous plant nucleic acid sequences, a geminivirus SIR, a virus Rep/RepA coding sequence, and a second geminivirus LIR.

28. The nucleic acid of claim 27, wherein the geminivirus is wheat dwarf virus or bean yellow dwarf virus.

29. The nucleic acid of claim 27, further comprising a sequence encoding a first sequence specific endonuclease that targets the first endogenous plant nucleic acid sequence.

30. (canceled)

31. The nucleic acid of claim 27, further comprising a second sequence that comprises, in order from 5′ to 3′, a third geminivirus LIR, a second repair template targeted to a second endogenous nucleic acid sequence within the plant, a second geminivirus SIR, a virus Rep/RepA coding sequence, and a fourth geminivirus LIR.

32-35. (canceled)

36. The nucleic acid of claim 27, wherein the nucleic acid comprises, in order from 5′ to 3′, the first geminivirus LIR, the first repair template, a second repair template targeted to a second endogenous nucleic acid sequence within the plant, the geminivirus SIR, the virus Rep/RepA coding sequence, and the second geminivirus LIR.

37. The nucleic acid of claim 36, further comprising a sequence encoding a first sequence specific endonuclease that targets the first endogenous plant nucleic acid sequence, and a sequence encoding a second sequence specific endonuclease that targets the second endogenous plant nucleic acid sequence.

38-42. (canceled)
